SEQUENCE LISTING (34 sequences)

SEQ ID NO: 1 - DNA, 223 bp, Human
tgacaaggtgccggattgacagccctggagactgaaatcctctatttatccacaggacaggtacagcacaggcagcgacagtgcgagctttccccacaccaccccgtccatgtgcctcaaccctgacctggagggaccacctctagaggtgagaggggatgttcagtctccaaggctcactcaatccttccgcctcagccgagactgccaacacacggggggc

SEQ ID NO: 2 - DNA, 18 bp, Human
agcgacagtgcgagcttt

SEQ ID NO: 3 - DNA, 20 bp, HUMAN
ggaaggattgagtgagcctt

SEQ ID NO: 4 - DNA, 17 bp, HUMAN
tgacgcatggcaacacc

SEQ ID NO: 5 - DNA, 17 bp, HUMAN
cgccttgacgcccgatt

SEQ ID NO: 6 - DNA, 23 bp, HUMAN
ttttgtgtgaatttcatgctggt

SEQ ID NO: 7 - DNA, 20 bp, HUMAN
ggtgtccacagcaccaaaaa

SEQ ID NO: 8 - DNA, 19 bp, HUMAN
ugacagcccuggagacuga

SEQ ID NO: 9 - DNA, 19 bp, HUMAN
ccacaggacagguacagca

SEQ ID NO: 10 - DNA, 26 bp, HUMAN
cagtctcggctgaggcggaaggattg

SEQ ID NO: 11 - DNA, 25 bp, HUMAN
gtgccggattgacagccctggagac

SEQ ID NO: 12 - DNA, 27 bp, HUMAN
ctctagaggtggtccctccaggtcagg

SEQ ID NO: 13 - DNA, 20 bp, HUMAN
aaggctcactcaatccttcc

SEQ ID NO: 14 - DNA, 25 bp, HUMAN
tgacagccctggagactgaaatcct

SEQ ID NO: 15 - DNA, 27 bp, HUMAN
ggacaggtacagcacaggcagcgacag

SEQ ID NO: 16 - DNA, 620 bp, HUMAN
aagcagtggtatcaacgcagagtacatgggagtgggctctgtaggggttcaacttaatcccacactgaccaggagctggcatcagctctgttgccccaccccttcattcttattgaaaatagcccaattgtgcagtgtgaaatctgccactaggtaacttttttttttaattcactgattacagctcgtttcaaattgtgtctgagctcctttttaaaggaaaaaggaaaaaaataaacaactttttttgtgtgaatttcatgctggtgacaaggtgccggattgacagccctggagactgaaatcctctatttatccacaggacaggtacagcacaggcagcgacagtgcgagctttccccacaccaccccgtccatgtgcctcaaccctgacctggagggaccacctctagaggtgagaggggatgttcagtctccaaggctcactcaatccttccgcctcagccgagactgccaacacacggggggccagtggcgctggtgatttttggtgctgtggacaccacctgtccacggggacctggactgacccccccaacctcatttcacccaggccgcgtagcccaccagatggtaacaccaacttttttttttttttt

SEQ ID NO: 17 - DNA, 336 bp, HUMAN
gggggtttgacaggcaacgaatatcgttattagacgctttttccataattgctgacatttatccagtgtttcacagtatttgctaatgtattaatgcagtcaatctctgagaggctgcacatgcctttttagccctggcttttatgggattctgaaattcatagacccacagatacttattccctgctggttgccgtagtgacagagctcagagataagccaagacagagcccttttgtgattgcttttcaggtgacattcagcggcagcttgagtagagtgtccttttatagttcctgtaattcagaaaatgataacagctttttgctgcttaga

SEQ ID NO: 18 - DNA, 573 bp, HUMAN
agatggttgtactgatggcttgtttttcattttttttgtgctttttggtccatctattaataaaaatgaaccccgttacagagtcaccatcatgtctcttctcaccaccctctgaatctgcattagccagtcaactagccctttcagcgtcatgtgaccagcgcgccccattcagcttggctggtgtcgtttcacatgacccaggctggccagtcgtcaggttgcaccgccctttggttcccgagcatgctgttttctctcagccttctctccaaccttaaccaaatcggcagcagccacctcgaccgcccacacattcctggccaatcagctcagctgtttatttaccaaatgtcttcacaacaactacagcagcagccttcggctaacaaaaaagcaggaaaaatccacaacacccccttcgccaaccaactaaatccaacgcaacatctggcaaaaccttttcagcaaattcttcctggccgtcagtccggcagcctcacctcaccatttctagcttgttgaaacccaaaactagtaagtttttcctgcttatacagtttactgctggtt

SEQ ID NO: 19 - DNA, 211 bp, HUMAN
ggatgcaaagatataaaagatagaagaacctagtaattgcctttaagatcagagcacaaagcattagtttggattttacaaggccagccagcaacaaaagagcagtaatttatgagctcagcgtaaaagaatagcataattgcattaaaaattaatgctaatgaacactaaaatggggaaggtggttatgatgaaagtttgtattcatctt

SEQ ID NO: 20 - DNA, 251 bp, HUMAN
cgccccctcctcaccattattgagaccaccaaagattccctgtccatgacctctgaggcggggttaccttgcttgtcggaccaaggcagcgatggttgtgatgatgatggcttggtgatatctgggtatggctcaggggaaacctttgactctaacctgccccctactgatgatgaagatttttacaccaccttctccttggtaacagataagagtctttccacttcaatcttcgaaggtggctacaaagc

SEQ ID NO: 21 - DNA, 203 bp, HUMAN
gaagaagctctgttcattaattcatgctcagcaaattgctaactaatttagttgcactcctctgcattaatggattattttataaaaatgttttccacagcccaagacctttggtgcatgcttttgagaggcttgaattctaatcaaccttaacagtaaattaggaatgtaatatctaaataataattgggatggcatatgga

SEQ ID NO: 22 - DNA, 248 bp, HUMAN
gctcagtagcttttcttaaaatagacagcctgtaaataaggtctgtgaactcaattgaaggtggctgtttctgaattagtcagccctcacaggctctcggcctacatgctagtacataaattgtccactttaccaccagacaagaaagattagagtaataaacacggggcattagctcagctagagaaacacaccagccgttacgcacacacaggattgccaagaactgttaacccaactctccagaa

SEQ ID NO: 23 - DNA, 266 bp, HUMAN
ccattttgcagccagcggagccattcacacaatcaccttctgttaattctatctgcaacatcaattaaattgtttgtagaaactaattgaatgtggcttaatcacacatcttaatagctttccacgctctgaactcgttgttaattccagtaataaaacgagatgagaacgctggctgggtaattagcgaggaggctggggaagaaattgctgtttgatggtgggtaataaaggcagccgtggtgaccgctctcctgagcagccgc

SEQ ID NO: 24 - DNA, 255 bp, HUMAN
tgccagggtttgagagctaattggaattagaataacatttctcacaggccattcatttgctcaggctgagtacaaattactatgcaagggaggctgagggttcctaatgatacaggataatgatgttttattcagattttaacgaggtgtaatcattctggttgagagagaaaaagattaagagggtgcaacaattaccaatttttacaactgattaatcgcaagccattacttgttcttggtcaaataggcatt

SEQ ID NO: 25 - DNA, 218 bp, HUMAN
agaatctacttacgttcaggatcactggtttcctgctcactctgctccttgttcttgtagaatgggaattttcgtgaaaagatgaagctctttttacgcttgtcattgaatgactgtgaaggagaaaaggcatggggcaaaaacaggggacgtctaattgttgtcacactcatgctgacaaaacaactagtttgtaaaagcacagagagctgtagtga

SEQ ID NO: 26 - DNA, 347 bp, HUMAN
agacctcctgggcacaacagtggcccactaataaaatgaaatgatagcatatgtgctttgtttaattttgatgtcttcataatgtattacacaattagattgatttgacaatttccattatgcttgataaaatatttattgagccctgaagatgataactcttatctaaccaccatttgttctacagatgtgacatatttaatatataagtaacatcactcagatctatgttccagttagcccattacattttatttgcagagtacagtgtaaagtcatcaataacgctttgatagatccctgtctgcccggtgcttgacaggtgcggagagagcttgctcacattc

SEQ ID NO: 27 - DNA, 284 bp, HUMAN
aaataaaacaaaagggcgagacaagagccagctgagagcaggatgaaatagcgttttttggcctgcatgtgtcctgcctgggtcaagaggattgtaaataaacacaagaatcacccaagtttcccaatcaacactttcagctccagtccgccatcttcccgcctgtaaagacttcaaactctccgctcccaccttctctggatgcctttccccaagccgtggactgatccagcgcatgtgggcgatttctttaactttctccccttcggtctttcatctttggg

SEQ ID NO: 28 - DNA, 243 bp, HUMAN
taattatttgatctgcagatgtaagtggaatttattgtaacaacaaatatagtgatgtcaaaacccaaccttggattaaagccggcagccaaaggggaagtgtattaatttccaacagcatctcaggccagagcctattagaacaagctattcttcaggcccccccatcagaattaatcattgggagttaatttgaagctcagacaagttgctgttaatttagtgcagggaggacgggatgga

SEQ ID NO: 29 - DNA, 222 bp, HUMAN
gagctggcttgctgggccacactgcagtcatgctgtttttgaattctctctctgttttgttctctgttctgtgctgttgtgtctccccgtgtctggtccccaggatccggcgctaagaaacagggcgaggacttagcggataatgacggggatgaagatgaaggtatttgggggctgcagggcgcggcggctggtgcatggcacagagcccctccccttctc

SEQ ID NO: 30 - DNA, 255 bp, HUMAN
cccttaaaggctgctaatgagaaatagagcctcattatgattttaaggatgagagaggaaggcttttaattaatgatatgtaaatggtcttgaattgattatgttagaatatcaagagagatgggtagaagctgtggtgtaatatattttaattaagaattaattaaaaatgatggcaatttcataaagcctcacagaaaatagtggaatttactttctgtcaggagtaaattagcctgtgattaaataaagggc

SEQ ID NO: 31 - DNA, 209 bp, HUMAN
gtcaggcagatctttggcgactacaagaccaccatctgcggcaagggcctgagcgcaacggtaacagggggccagaaggggaggggttccagaggccaacatcaagctcattctttggagcgggtgtgtcattgtttgggaaaatggctaggacatcccgacaaggtgatcatcctcaggattttgtggcaataacaaggggtggggga

SEQ ID NO: 32 - DNA, 217 bp, HUMAN
gaattcagcatgggcccattatcgactgctttttcgtgtgttgtaaaaggagtgagacctcagggaggcttggagcctcctcccgtgtaatgtccaagtcagatttgaaggactggtggcaggaaagctttggccttggggtgtgaattctaagaaatagaggcacaattaagtttaaaaaattcaaggactttgagcttgataatgaaagattttt

SEQ ID NO: 33 - DNA, 232 bp, HUMAN
gaataataagcatcataaaatctaatcagatttataagaggaattgattgttgtggcaggaggagagggtggaaaataagctgaaatttaagtgttaatttttatgctaatccacagtggcttcatatgaagcaattttgttctgtgccacaaatataaaattgtcccttgcagagaaatgtcagcttgctagacctgaaagcagtaatttattaggtatgtttctattagt

SEQ ID NO: 34 - DNA, 269 bp, HUMAN
gccgccaccaacacctttctttattccgcccaaacctcaccaaggggaattcgaaacaactgtggtgataaatccgcctgcagataattggcaagaaaacaacacttgtgcagtatataacgttttatggttgtttcatgtactttattataatactaatgagcagttatatatcaggagctaactgcacgccggattagcttgttagcatgatatgaaatggcagagcaaacagtccaggcccctcgctccctgcatgcctcccccac

(19) United States
(12) Patent Application Publication
(10) Pub. No.: US 2011/0250608 A1
(43) Pub. Date: Oct. 13, 2011

(54) USE OF ULTRACONSERVED RNA IN DETECTION AND TREATMENT OF CANCER

(75) Inventors: Tushar Patel, Ponte Vedra Beach, FL (US); Chiara Braconi, Columbus, OH (US)

(73) Assignee: THE OHIO STATE UNIVERSITY, Columbus, OH (US)

(21) Appl. No.: 13/084,279

(22) Filed: Apr. 11, 2011

Related U.S. Application Data
(60) Provisional application No. 61/322,608, filed on Apr. 9, 2010.

(51) Int. Cl. C12Q 1/68; C07H 21/04; C07H 21/02; G01N 33/53; C12N 15/63

(52) U.S. Cl. 435/6.14; 436/64; 435/320.1; 536/24.5; 536/23.1

(57) ABSTRACT

A method of analyzing a biological specimen to detect cancer in a subject, involving determining an ultraconserved RNA in the specimen and comparing the expression level to a control. The ultraconseved RNA may be uc.338 and may be used to detect hepatocellular cancer. Transcript RNA encoding ultraconserved RNA may also be used to detect cancer. Anti-cancer compositions and tumor-suppressing agents for treating cancer are also provided.

### CROSS-REFERENCE TO RELATED APPLICATIONS

This application claims priority of U.S. provisional application Ser. No. 61/322,608, filed Apr. 9, 2010, the disclosure of which is incorporated herein by reference as if fully recited herein.

### TECHNICAL FIELD

Embodiments relate to methods, compositions, and systems for detecting and treating cancer in a subject, particularly to method's, compositions, and systems for detecting and regulating ultraconserved RNA or transcript RNA.

### BACKGROUND

Hepatocellular carcinogenesis involves a complex interaction of genes resulting in variable modulation of key pathways involved in tumor cell growth. Using molecular techniques for global genomic profiling, the transcriptome in hepatocellular cancers (HCC) has been described, and several genes that are differentially activated have been identified. The major focus of attention in these efforts has been on the characterization of expression of protein-coding genes and their use for determining clinical outcomes. However, the majority of the human genome consists of non-protein-coding RNA (ncRNA), some of which is transcribed. Increasing evidence points to an important functional or regulatory role of ncRNA in cellular processes, as well as a contribution of aberrant ncRNA expression to disease phenotypes.

Along with the highly abundant transfer and ribosomal RNAs, ncRNAs include microRNAs that modulate mRNA expression, small nucleolar RNAs that guide chemical modification of RNA-molecules, small interfering RNAs (siRNAs) that account for the interference pathway, piwiRNAs that are linked to transcriptional gene silencing of retrotransposons and long non coding RNAs whose role is still unknown. The role played by the non-coding RNA genome in malignant transformation and tumor growth in HCC is being increasingly recognized. Researchers have recently provided data from profiling studies in which several microRNAs were identified and shown to be involved in the modulation of cell proliferation and apoptosis. Recent studies revealing the presence of several hundred long transcribed ncRNAs raise the possibility that many ncRNAs contributing to cancer remain to be discovered. Other than microRNAs, however, only a handful of ncRNA have been implicated in hepatocarcinogenesis. For the most part, the function of these ncRNAs is unknown. Sequence conservation across species has been postulated to indicate that a given ncRNA may have a cellular function. A genome-wide survey identified several hundred ncRNAs with a size greater than 200 bp that showed a remarkable conservation with 100% identity across the human, mouse and rat genomes. These highly conserved long ncRNAs have been named ultraconserved regions, and are conserved across many other species as well, with 99% of these genes showing high levels of conservation within the dog, 97% within the chicken and 67% within the fugu genomes. Their wide distribution in the genome and lack of natural variation in the human population suggested that these ncRNA genes have a biological function which is essential for normal cells. However, the function of these ultraconserved genes remains controversial and unknown.

### SUMMARY

Embodiments relate to a method of analyzing a biological specimen to detect cancer in a subject, comprising the steps of: (a) determining the expression level of a RNA sequence in the specimen; and (b) comparing the expression level to a control, wherein a pre-identified difference between the determined expression level and the control is indicative of cancer in the subject. In some embodiments, the RNA sequence is uc.338. In various embodiments, the RNA sequence is selected from the groups consisting of uc.338, uc.24, uc.189, uc.134, uc.378, uc.349, uc.78, uc.233, uc.262, uc.331, uc.136, and uc.246. In various other embodiments, the RNA sequence is selected from the groups consisting of uc.110, uc.473, uc.275, uc.477, uc.269, uc.448, and uc.20. In some embodiments, the RNA sequence may be TUC338 or any other transcript RNA that is capable of encoding an ultraconserved RNA selected from the group consisting of uc.338, uc.24, uc.189, uc.134, uc.378, uc.349, uc.78, uc.233, uc.262, uc.331, uc.136, and uc.246. In various other embodiments, the RNA sequence is one that is capable of encoding an ultraconserved RNA selected from the group consisting of uc.110, uc.473, uc.275, uc.477, uc.269, uc.448, and uc.20.

In some embodiments, the subject is a mammal. In specific embodiments, the subject is a human.

In exemplary embodiments, the cancer is a hepatocellular cancer. In specific embodiments, the subject is suspected of having HCC or is at risk for HCC. In some embodiments, the control is a non-malignant hepatocyte.

In some embodiments, the expression of uc.338 is determined by an amplification assay or a hybridization assay.

Embodiments are also directed towards an isolated small inhibitory ribonucleic acid (“siRNA”) molecule that inhibits expression of a nucleic acid molecule encoding an ultraconserved RNA molecule. In various embodiments, the ultraconserved RNA molecule is selected from the group consisting of uc.338, uc.24, uc.189, uc.134, uc.378, uc.349, uc.78, uc.233, uc.262, uc.331, uc.136, and uc.246. In other embodiments, the ultraconserved RNA is selected from the group consisting of uc.110, uc.473, uc.275, uc.477, uc.269, uc.448, and uc.20. In embodiments, the ultraconserved RNA is uc.338. In various embodiments, the siRNA comprises an isolated nucleic acid comprising the nucleotide sequence of SEQ ID NO: 6. In other embodiments, the siRNA comprises an isolated nucleic acid comprising the nucleotide sequence of SEQ ID NO: 7.

Various embodiments are directed towards an anti-cancer composition comprising an antisense oligonucleotide, ribozyme, siRNA, or any combination thereof, that binds to uc.338.

Other embodiments are directed to a tumor-suppressing agent comprising as an active ingredient, at least one of: a double stranded RNA complementary to a transcript of an ultraconserved RNA gene; a DNA encoding a double-stranded RNA complementary to a transcript of an ultraconserved RNA gene; and a vector carrying, as an insert, a DNA encoding a double-stranded RNA complementary to a transcript of an ultraconserved RNA gene. In various embodiments, the ultraconserved RNA gene is selected from the group consisting of uc.338, uc.24, uc.189, uc.134, uc.378, uc.349, uc.78, uc.233, uc.262, uc.331, uc.136, and uc.246. In some embodiments, the ultraconserved RNA gene is selected from the group consisting of uc.110, uc.473, uc.275, uc.477, uc.269, uc.448, and uc.20. In various embodiments, the ultraconserved RNA is uc.338. In various embodiments the transcript is TUC338.

### DESCRIPTION OF THE SEQUENCES

The various embodiments can be more fully understood from the following detailed description and the accompanying sequence descriptions, which form a part of this application.

TABLE 1

| SEQ | | |
|---|---|---|
| ID | | |
| NO: | Name | Sequence |
| 1 | uc.338 (DNA) | TGACAAGGTGCCGGATTGACAGCCCTGGAG |
| | | ACTGAAATCCTCTATTTATCCACAGGACAGGT |
| | | ACAGCACAGGCAGCGACAGTGCGAGCTTTC |
| | | CCCACACCACCCCGTCCATGTGCCTCAACCC |
| | | TGACCTGGAGGGACCACCTCTAGAGGTGAG |
| | | AGGGGATGTTCAGTCTCCAAGGCTCACTCAA |
| | | TCCTTCCGCCTCAGCCGAGACTGCCAACACA |
| | | CGGGGGGC |
| 2 | uc.338(for | AGCGACAGTGCGAGCTTT |
| | RTpcr): | |
| 3 | uc.338(for | GGAAGGATTGAGTGAGCCTT |
| | RTpcr): | |
| 4 | uc.338-F: | TTTTGTGTGAATTTCATGCTGGT |
| 5 | uc.338-R: | GGTGTCCACAGCACCAAAAA |
| 6 | siRNA-1 (RNA) | UGACAGCCCUGGAGACUGA |
| 7 | siRNA-2 (RNA) | CCACAGGACAGGUACAGCA |
| 8 | TUC338 | AAGCAGTGGTATCAACGCAGAGTACATGGGAG |
| | | TGGGCTCTGTAGGGGTTCAACTTAATCCCACA |
| | | CTGACCAGGAGCTGGCATCAGCTCTGTTGCCC |
| | | CACCCCTTCATTCTTATTGAAAATAGCCCAAT |
| | | TGTGCAGTGTGAAATCTGCCACTAGGTAACTT |
| | | TTTTTTTTAATTCACTGATTACAGCTCGTTTC |
| | | AAATTGTGTCTGAGCTCCTTTTTAAAGGAAAA |
| | | AGGAAAAAAATAAACAACTTTTTTTGTGTGAA |
| | | TTTCATGCTGGTGACAAGGTGCCGGATTGACA |
| | | GCCCTGGAGAGACTGAAATCCTCTATTTATCC |
| | | ACAGGACAGGTACAGCACAGGCAGCGACAGTG |
| | | CGAGCTTTCCCCACACCACCCCGTCCATGTGC |
| | | CTCAACCCTGACCTGGAGGGACCACCTCTAGA |
| | | GGTGAGAGGGGATGTTCAGTCTCCAAGGCTCA |
| | | CTCAATCCTTCCGCCTCAGCCGAGACTGCCAA |
| | | CACACGGGGGGCCAGTGGCGCTGGTGATTTTT |
| | | GGTGCTGTGGACACCACCTGTCCACGGGGACC |
| | | TGGACTGACCCCCCCAACCTCATTTCACCCAG |
| | | GCCGCGTAGCCCACCAGATGGTAACACCAACT |
| | | TTTTTTTTTTTTTT |

SEQ ID NOS: 2-5 are examples of primers and primer sequences that can be used for PCR amplification. However, it is appreciated that various alternative primers may be designed.

Other features and advantages will be apparent from the following detailed description, and from the claims.

### DRAWINGS

A better understanding of the embodiments will be obtained from a reading of the following detailed description and the accompanying drawings in which:

FIG. 1. ucRNAs are aberrantly expressed in malignant hepatocytes. Panel A: Genome-wide expression profiling was performed using a custom microarray in HepG2 cells and normal human hepatocytes (HH). 56 ucRNAs were aberrantly expressed in malignant hepatocytes with a p value<0.05, with twelve ucRNAs increased and 7 decreased by greater than 2-fold as shown in Table 2 below:

TABLE 2

| | FOLD CHANGE | FOLD CHANGE, | Parametric |
|---|---|---|---|
| ucRNA | HCC vs HH | log2 | p-value |
| uc.338+ | 6.99 | 2.81 | 0.0001277 |
| uc.24 + A | 4.75 | 2.25 | 0.0466492 |
| uc.189+ | 4.23 | 2.08 | 0.0000211 |
| uc.134 + A | 3.78 | 1.92 | 0.000059 |
| uc.378+ | 2.98 | 1.57 | 0.0000213 |
| uc.349+ | 2.96 | 1.57 | 0.0001016 |
| uc.78+ | 2.69 | 1.43 | 0.0001255 |
| uc.233 + A | 2.37 | 1.24 | 0.0008148 |
| uc.262 + A | 2.30 | 1.20 | 0.0056489 |
| uc.331+ | 2.24 | 1.17 | 0.0000605 |
| uc.136+ | 2.18 | 1.12 | 0.0020847 |
| uc.246+ | 2.00 | 1.00 | 0.0001246 |
| uc.47+ | 1.96 | 0.97 | 0.008174 |
| uc.204 + A | 1.94 | 0.96 | 0.0225475 |
| uc.139+ | 1.92 | 0.94 | 0.0406478 |
| uc.362 + A | 1.88 | 0.91 | 0.0108186 |
| uc.10 + A | 1.86 | 0.90 | 0.0021238 |
| uc.339+ | 1.82 | 0.86 | 0.0007036 |
| uc.420+ | 1.76 | 0.81 | 0.0011707 |
| uc.412 + A | 1.75 | 0.81 | 0.0070073 |
| uc.234+ | 1.74 | 0.80 | 0.0024131 |
| uc.372+ | 1.70 | 0.77 | 0.0379089 |
| uc.44 + A | 1.60 | 0.67 | 0.0048444 |
| uc.456 + A | 1.54 | 0.62 | 0.0114 |
| uc.190 + A | 1.53 | 0.61 | 0.0081303 |
| uc.177 + A | 1.51 | 0.59 | 0.0115514 |
| uc.392 + A | 1.49 | 0.57 | 0.0195016 |
| uc.278+ | 1.46 | 0.54 | 0.041165 |
| uc.470 + A | 1.44 | 0.53 | 0.0081265 |
| uc.213+ | 1.41 | 0.50 | 0.0249108 |
| uc.117 + A | 1.36 | 0.44 | 0.0153249 |
| uc.1+ | 1.31 | 0.39 | 0.0414189 |
| uc.346+ | 1.31 | 0.39 | 0.0463969 |
| uc.475+ | −1.24 | −0.31 | 0.0418998 |
| uc.95+ | −1.30 | −0.38 | 0.0363832 |
| uc.462+ | −1.32 | −0.40 | 0.0183523 |
| uc.16+ | −1.38 | −0.47 | 0.0491905 |
| uc.369+ | −1.40 | −0.49 | 0.0473564 |
| uc.100+ | −1.42 | −0.51 | 0.0076957 |
| uc.48+ | −1.47 | −0.55 | 0.0418279 |
| uc.305+ | −1.55 | −0.63 | 0.0114024 |
| uc.88+ | −1.63 | −0.70 | 0.0015275 |
| uc.325 + A | −1.66 | −0.73 | 0.0068587 |
| uc.388+ | −1.66 | −0.73 | 0.0018223 |
| uc.153+ | −1.79 | −0.84 | 0.00126 |
| uc.411 + A | −1.87 | −0.91 | 0.0059916 |
| uc.18+ | −1.88 | −0.91 | 0.0105249 |
| uc.128 + A | −1.89 | −0.92 | 0.0009112 |
| uc.33 + A | −1.92 | −0.94 | 0.011849 |
| uc.110 + A | −2.06 | −1.04 | 0.000097 |
| uc.473+ | −2.12 | −1.09 | 0.0000668 |
| uc.275+ | −2.23 | −1.16 | 0.0001138 |
| uc.477+ | −2.44 | −1.29 | 0.0001645 |
| uc.269 + A | −2.71 | −1.44 | 0.0008003 |
| uc.448+ | −3.15 | −1.66 | 0.0000296 |
| uc.20 + A | −3.42 | −1.78 | 0.0000035 |

The ratio of expression of these ucRNAs in malignant HepG2 cells relative to HH is plotted against the p-value. Selected ucRNAs with a greater than 3-fold change in expression are annotated. Panel B: The genomic locations of the ucRNA as exonic, non exonic or possibly exonic relative to protein-coding genes is depicted for all ucRNAs and for the group of ucRNAs that are aberrantly expressed in malignant hepatocytes. Selective enrichment of a specific group of ucRNA based on relationship to known protein-coding genes was not observed.

FIG. 2. uc.338 is overexpressed in HCC cells lines. Panel A: Schematic representation of the partial exonic location of uc.338 within the PCBP2 gene. The exons of PCBP2 are indicated by dark grey boxes, while uc.338 is depicted as the light grey box. The location of the uc.338 forward (F) and reverse (R) primers used for real-time-PCR and probe used for in situ hybridization are shown. With the exception of the forward primer, these are located within the PCBP2 intronic region. Of the siRNAs targeting uc.338, siRNA-1 is entirely intronic while siRNA-2 overlaps a few nucleotides of the coding sequence of PCBP2. F′ and R′ indicate primers used for detection of PCBP2. Panel B: RNA was extracted from different cell lines and uc.338 expression evaluated by quantitative real-time-PCR. The expression of uc.338 was normalized to that of RNU6. Bars represent the mean and SEM of 4 replicates. uc.338 expression was increased in all HCC cell lines compared to normal human hepatocytes (HH). * p<0.05 relative to HH. Non-malignant epithelial cells (hatched bars): HE: hepatocytes, BE: biliary epithelia, PE: prostatic epithelia. Malignant cells (solid bars): HCC: hepatocellular cancer, CCA: cholangiocarcinoma, PaC: Pancreatic cancer, CRC: colorectal cancer, PC: prostate cancer, BC: breast cancer.

FIG. 3. Representation of the different classes of staining after in situ hybridization with locked nucleic acid (LNA)-anit-uc.338.

FIG. 4. uc.338 is over-expressed in human HCC tissues. uc.338 expression was evaluated in a total of 221 HCCs, 72 cases of non cirrhotic and 97 cases of cirrhotic adjacent liver tissues. Paraffin-embedded, formalin-fixed liver tissues were incubated with LNA-anti-uc.338. Panel A: uc.338 expression was classified as negative, weak, moderate, or strong based on the percentage of cells with detectable staining for uc.338. The proportion of cases of HCC, cirrhotic liver, or non-cirrhotic liver within each class is depicted in the columns. Panel B: The mean and 95% confidence intervals of uc.338 expression in non-cirrhotic liver, cirrhotic liver and HCC tissues is shown. *p<0.05. Panel C: uc.338 expression was compared between 156 HCCs and their corresponding adjacent liver tissues. Picture of representative cases are shown. Panel D: An expression score was derived as the ratio of the difference in expression between HCC and adjacent liver tissues to the standard deviation of uc338 in all tissues, and plotted with the size of the bubble representing the number of cases.

FIG. 5. Nuclear expression of uc.338 in HCC cells. Panel A: Paraffin-embedded, formalin-fixed liver tissues were incubated with LNA probe anti-uc.338. uc.338 was frequently detected in nuclei of HCC in situ. Panel B: RNA was extracted from the nuclear and the cytoplasmic fraction of HCC cells and uc.338 expression evaluated by real-time-PCR. Bars represent the mean and SEM of relative expression of uc.338 from two experiments performed in four replicates. * p<0.05.

FIG. 6. uc.338 and PCBP2 are independently regulated. Panel A: Expression of uc.338 is plotted against PCBP2 mRNA expression in samples of normal and HCC cell lines. There is no linear correlation between uc.338 and PCBP2 gene expression (P=0.08). Panel B: HepG2 cells were transfected with siRNA against PCBP2 or siRNA control. Bar represents the mean of 2 independent experiments performed in four replicates. *p<0.05 compared to control siRNA. Panel C: The expression of uc.338 was decreased in HepG2 and Huh-7 cells using two different siRNAs against uc.338. After 48 hours RNA was collected and uc.338 and PCBP2 expression evaluated by real time PCR. Bars represent the mean and SEM of 3 experiments performed in 4 replicates. *p<0.05 relative to siRNA control. These data indicate that uc.338 does not regulate the expression of PCBP2 and that the expression of uc.338 is independent of PCBP2.

FIG. 7. Schematic Representation of uc.338 and PCBP2 gene. The exons of PCBP2 are indicated by dark gray boxes, and uc.338 is depicted as the light gray box. Primers used for the 5′ RACE were as follows: the antisense intronic (ASI) primer recognized the antisense transcript, and the sense intronic (SI) and nested SI (nSI) primers recognized the sense transcript. The sense exonic (SE) primer recognized PCBP2. Primers used for the 3′ RACE were as follows: the ASI and nASI primers were used to characterize the 3′ UTR of the TUC338 sense transcript, and the antisense exonic (ASE) primer was used to characterize the 3′ UTR of PCBP2.

FIG. 8. 5′RACE. HepG2 RNA was retrotranscribed by using the SMARTer RACE cDNA Amplification Kit (Clonetech). The 5′ RACE-ready cDNA was then amplified by PCR using the Universal Primer Mix (UPM) that recognized the SMARTer oligonucleotide added at the 5′ end and the specific gene primers. The SE primer was used as a control to sequence the 5′ end of PCBP2 (band 1). Given the multiple bands obtaind with the SI primer, the PCR product was further amplified witht the nested UPM (nUPM) and nSI primers producing a defined band (band 2). The sequence obtained from the DNA extracted from band 2 is shown. Underline, uc.338 sequence identified by Bejerano et al. (1); bold, exonic sequence; italic, nSI sequences; DNA marker, 1-kb DNA ladder (Promega, Madison, Wis.).

FIG. 9. 3′ RACE. HepG2 RNA was polyadenylated and retrotranscribed by using the SMARTer RACE cDNA Amplification Kit. The 3′ RACE-ready cDNA was then amplified by PCR using the UPM and specific gene primers. The ASE primer was used as a control to sequence the 3′ end of PCBP2 (band 3). 3′ RACE-ready cDNA was amplified by a first PCR using the ASI primer, and a second nested PCR with the nASI primer (band 4). The sequence obtained from the DNA extracted from band 4 is shown. Underline shows uc.338(1); capital character, exonic sequence; italic, nSI sequences, DNA marker, 1-kb and 100-bp DNA ladder (Promega, Madison, Wis.).

FIG. 10. Cloning of the transcript including uc.338. Panel A: Schematic representation of the transcript including uc.338 (TUC338) in relation to PCBP2 gene. By performing 5′ and 3′ rapid amplificiation of cDNA ends (RACE), we identified 237 nt upstream and 130 nt downstream of the ultraconserved region. The complete sequence of the TUC338 transcript is reported. Panel B: Northern blotting analysis for TUC338 and PCBP2 was performed. TUC338 was expected to be 590 nt long, and PCBP2 was around 1,200 nt long.

FIG. 11. TUC338 modulates cell growth in HCC cells. Panel A: HepG2 and Huh-7 cells were transfected with siRNAs against TUC338 or control siRNA for 48 hours and then plated (200,000 viable cells/well) in 6-well plates. After 24, 48 and 72 hours cells were counted by trypan blue staining. Mean values of three independent experiments with SEM are represented. * p<0.05 compared to siRNA control. Cell viability following transfection with siRNA-1 was not significantly different from that with siRNA-2 (p>0.05 at each time point in each of the cell lines). Panel B: HepG2 cells were transfected with siRNA-2 anti-TUC338 or control siRNA for 48 hours, and analysis of cell cycle distribution was performed by flow cytometry. Compared to controls there was a reduction of 30% in S phase and of 12% in G2/M phase for cells transfected with siRNA against TUC338. Bars represent the mean and SEM of three experiments. *p<0.05 compared to controls. Panel C: HepG2 cells were transfected with siRNAs anti-TUC338 or control siRNA by nuclear transfection for 48 hours and then plated in agar in 96-well plates and anchorage-independent growth assessed fluorometrically after 7 days. Bars represent the mean and SEM of 2 experiments performed in seven replicates. *p<0.05.

FIG. 12. TUC338 modulates cell growth in mouse hepatocytes. BNL-CL.2 are nonmalignant embryonic mouse hepatocytes. BNL-SV A.8 cells are derived from BNL-CL.2 following SV40 transformation, and exhibit transformed cell growth. Panel A: TUC338 expression was assessed by real-time-PCR and normalized to that of RNU6. Bars represent the mean and standard error of 3 experiments. *p<0.05. Panel B: BNL-CL.2 and BNL-SVA.8 were plated in soft agar and colonies counted after 4 weeks. Representative pictures are shown along with the mean and SEM of three independent experiments. *p<0.05. Panel C: BNL-CL.2 and BNL-SV A.8 cells were plated in 6-well plates and cell number counted by trypan blue staining at different time points. Mean and SEM derived from three independent experiments are shown. *p<0.05 compared to BNL-CL.2. Panel D: BNL-SV A.8 cells were transfected with siRNA-1 anti-TUC338 or siRNA control for 48 hours. Bars represent mean and SEM of four replicates. At the indicated times, cells were counted after trypan blue staining. Mean and SEM from three independent experiments are represented. *p<0.05 compared to siRNA control.

FIG. 13. TUC338 expression modulates expression of cell-cycle regulatory proteins. HepG2 and Huh7 cells were serum-starved for 48 hours before transfection with either control siRNA or anti-TUC338 siRNA. Cells were collected 48 hours after transfection, and protein lysates were obtained. Lysates were also obtained from untransfected cells and normal human hepatocytes (HH). Western blotting was performed for the indicated cell-cycle-associated proteins, and their expression was quantitated by densitometry and normalized to that of vinculin. The expression relative to cells transfected with a control siRNA is reported along with representative immunoblots. PCNA is the acronym for proliferating cell nuclear antigen.

### DESCRIPTION

Embodiments are based, in part, on the discovery of a functional role for long non-coding RNA genes in cell growth modulation. Embodiments exploit the effects of ucRNAs on tumor cell growth, and demonstrate these RNA genes are useful for studying, diagnosing, and treating cancers, particularly liver cancers.

The RNA uc.338 is exonic, and represents an alternatively spliced exon of PCBP2, an RNA binding protein involved in mRNA processing. Interestingly, exonic ultraconserved regions are frequently associated with RNA processing such as RNA binding or RNA splicing, suggesting a potential role as regulators of RNA. Despite the exonic location of uc.338 within PCBP2, the expression of these two genes is independent. Advantageously, the examples below demonstrate an important role for uc.338 in human cancer. Various embodiments exploit this novel ucRNA gene as a diagnostic marker and a therapeutic target for HCC.

Unless otherwise defined, all technical and scientific terms used herein have the same meaning as commonly understood by one of ordinary skill in the art to which these embodiments pertain. Although methods and materials similar or equivalent to those described herein can be used in the practice or testing of various embodiments, suitable methods and materials are described below. All publications, patent applications, patents, and other references mentioned herein are incorporated by reference in their entirety for all purposes. In case of conflict, the present specification, including definitions, will control. In addition, the materials, methods, and examples are illustrative only and not intended to be limiting.

The section headings used herein are for organizational purposes only and are not to be construed as limiting the described subject matter in any way. It will be appreciated that there is an implied “about” prior to metrics such as temperatures, concentrations, and times discussed in the present teachings, such that slight and insubstantial deviations are within the scope of the present teachings herein. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the foregoing general description and the following detailed description are exemplary and explanatory only and are not restrictive of the invention. The articles “a” and “an” are used herein to refer to one or to more than one (i.e., to at least one) of the grammatical object of the article. By way of example, “an element” means one element or more than one element.

As used herein, “cancer” refers to all types of cancers, or neoplasms or benign or malignant tumors.

The phrase “detecting a cancer” or “diagnosing a cancer” refers to determining the presence or absence of cancer or a precancerous condition in an animal. “Detecting a cancer” also can refer to obtaining indirect evidence regarding the likelihood of the presence of precancerous or cancerous cells in the animal or assessing the predisposition of a patient to the development of a cancer. Detecting a cancer can be accomplished using the methods of this invention alone, in combination with other methods, or in light of other information regarding the state of health of the animal.

The term “mammal” for purposes of treatment or diagnosis refers to any animal classified as a mammal, including humans, domestic and farm animals, and zoo, sports, or pet animals, such as dogs, horses, cats, cows, etc. In exemplary embodiments, a mammal is a human.

A “tumor,” as used herein, refers to all neoplastic cell growth and proliferation, whether malignant or benign, and all precancerous and cancerous cells and tissues.

Nucleic acid: a nucleic acid may include any polymer or oligomer of pyrimidine and purine bases, preferably cytosine, thymine, and uracil, and adenine and guanine, respectively. The present invention contemplates any deoxyribonucleotide, ribonucleotide or peptide nucleic acid component, and any chemical variants thereof, such as methylated, hydroxymethylated or glycosylated forms of these bases, and the like. The polymers or oligomers may be heterogenous or homogenous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

The detection probe may be comprised of naturally occurring or synthetic RNA, DNA, or other oligonucleotides such as an analog of a naturally occurring nucleic acid. At least one locked nucleic acid (LNA) molecule may also be included in the detection probe. A LNA can include a modified RNA nucleotide, for example, in which the ribose moiety of the LNA is modified with an extra bridge connecting the 2′ and 4′ carbons. The bridge may lock the ribose in a 3′-endo structural conformation, thereby enhancing base stability and backbone pre-organization. The LNA may be labeled with a fluorophore.

As used herein, the term “subject” is intended to include humans and non-human animals. The term “non-human animals” includes all.vertebrates, e.g., mammals and non-mammals, such as non-human primates, pigs, chickens and other birds, mice, dogs, cats, cows, and horses.

Sequencing: The term sequencing refers to determining the order of nucleotides (base sequences) in a nucleic acid sample, e.g. DNA or RNA.

Primers: in general, the term primers refer to DNA strands which can prime the synthesis of DNA. DNA polymerase cannot synthesize DNA de novo without primers: it can only extend an existing DNA strand in a reaction in which the complementary strand is used as a template to direct the order of nucleotides to be assembled. Herein, the synthetic oligonucleotide molecules which are used in a polymerase chain reaction (PCR) are referred to as primers. An “SNP-specific primer” is a primer appropriate for oligonucleotide extension at an SNP marker.

DNA amplification: the term DNA amplification will be typically used to denote the in vitro synthesis of double-stranded DNA molecules using PCR. It is noted that other amplification methods exist and they may be used without departing from the gist.

In various embodiments, DNA is to be provided. This can be done by methods known in the art per se. The isolation of DNA is generally achieved using common methods in the art such as the collection of tissue from a member of the population, DNA extraction (for instance using the Q-Biogene fast DNA kit), quantification and normalisation to obtain equal amounts of DNA per sample. The DNA can be from a variety of sources (Genomic, RNA, cDNA, BAc, YAC etc.) and organisms (human, mammal, plant, microorganisms, etc.). The isolated DNA may be pooled.

Also provided are various kits for performing the methods provided herein. Additionally, the kit may include instructional materials for performing various methods presented herein. These instructions may be printed and/or may be supplied, without limitation, as an electronic-readable medium, such as a floppy disc, a CD-ROM, a DVD, a Zip disc, a video cassette, an audiotape, and a flash memory device. Alternatively, instructions may be published on an internet web site or may be distributed to the user as an electronic mail. When a kit is supplied, the different components can be packaged in separate containers. Such packaging of the components separately can permit long term storage without losing the active components' functions.

In another aspect, the embodiments feature methods of treating a subject who has a disease characterized by abnormal expression of uc.338, as described herein. The methods include administering an inhibitor of uc.338 or TUC338 activity to the subject. In some embodiments, the inhibitor of uc.338 or TUC338 activity can include, for example, one or more of an antisense nucleic acid, a small interfering nucleic acid, etc. The embodiments additionally feature methods of treating a subject having a disease characterized by aberrant cellular proliferation or differentiation, e.g., as described herein. The methods include administering one or more inhibitors of uc.338 activity. In some embodiments, the inhibitor of uc.338 or TUC338 activity includes a nucleic acid molecule described herein.

The embodiments also provide kits including one or more compounds that selectively bind to an ultraconserved nucleic acid molecule (e.g. uc.338) as described herein, and instructions for use.

Embodiments include methods for detecting the presence of an uc.338 or TUC338 nucleic acid molecule in a sample. The method includes contacting the sample with a nucleic acid probe or primer that selectively hybridizes to the nucleic acid molecule, and determining whether the nucleic acid probe or primer binds to a nucleic acid molecule in the sample. In some embodiments, the sample comprises RNA molecules and is contacted with a nucleic acid probe. The invention also includes a kit comprising a compound that selectively hybridizes to an ultraconserved nucleic acid molecule (e.g. uc.338), and instructions for use.

Various embodiments also provide methods for identifying compounds that modulate the expression or activity of a nucleic acid described herein. The method includes contacting the nucleic acid with a test compound; and determining an effect of the test compound on the expression or activity nucleic acid, to thereby identify a compound that modulates the expression or activity of the nucleic acid.

In another aspect, the invention includes transgenic animals, e.g., animals at least some of whose somatic and germ cells comprise at least one uc.338 transgene.

Also within the embodiments is the use of uc.338, TUC338, and/or any of the inhibitors of uc.338 or TUC338 activity described herein, e.g., an antisense nucleic acid, a small interfering nucleic acid, or ribozyme, in the manufacture of a medicament for the treatment or prevention of disorders associated with aberrant cellular proliferation. The medicament can be used in a method for treating or preventing disorders associated with aberrant cellular proliferation (e.g., cancer) in a patient suffering from or at risk for a disorder associated with aberrant cellular proliferation.

Further, within the embodiments is the use of uc.338, TUC338, and/or any of the enhancers of uc.338 or TUC338 activity described herein, e.g., uc.338 nucleic acids, or active fragments thereof, in the manufacture of a medicament for the treatment or prevention of disorders associated with aberrant cellular differentiation and/or proliferation. The medicament can be used in a method for treating or preventing disorders associated with aberrant cellular differentiation and/or proliferation in a patient suffering from or at risk for a disorder associated with aberrant cellular differentiation and/or proliferation.

Also within the invention are uc.338 and TUC338 nucleic acids, antisense nucleic acids, and small interfering nucleic acids for use in treating disorders associated with aberrant cellular differentiation and/or proliferation.

### Pharmaceutical Formulations

The compositions of this invention can be formulated and administered to inhibit a variety of disease states by any means that produces contact of the active ingredient with the agent's site of action in the body of a mammal. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

Pharmaceutical compositions for use in accordance with the present invention may be formulated in conventional manner using one or more physiologically acceptable carriers or excipients. The therapeutic compositions of the invention can be formulated for a variety of routes of administration, including systemic and topical or localized administration. For systemic administration, injection is preferred, including intramuscular, intravenous, intraperitoneal, and subcutaneous. For injection, the therapeutic compositions of the invention can be formulated in liquid solutions, preferably in physiologically compatible buffers such as Hank's solution or Ringer's solution. In addition, the therapeutic compositions may be formulated in solid form and redissolved or suspended immediately prior to use. Lyophilized forms are also included.

For oral administration, the therapeutic compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active agent. For buccal administration the therapeutic compositions may take the form of tablets or lozenges formulated in a conventional manner. For administration by inhalation, the compositions for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflate or may be formulated containing a powder mix of the therapeutic agents and a suitable powder base such as lactose or starch.

The therapeutic compositions may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

In addition to the formulations described previously, the therapeutic compositions may also be formulated as a depot preparation. Such long acting formulations may be administered by implantation (for example subcutaneously or intramuscularly) or by intramuscular injection. Thus, for example, the therapeutic compositions may be formulated with suitable polymeric or hydrophobic materials (for example as an emulsion in an acceptable oil) or ion exchange resins, or as sparingly soluble derivatives, for example, as a sparingly soluble salt.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For topical administration, the compositions of the invention are formulated into ointments, salves, gels, or creams as generally known in the art. A wash solution can be used locally to treat an injury or inflammation to accelerate healing. For oral administration, the therapeutic compositions are formulated into conventional oral administration forms such as capsules, tablets, and tonics.

The therapeutic compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

A composition of the present invention can also be formulated as a sustained and/or timed release formulation. Such sustained and/or timed release formulations may be made by sustained release means or delivery devices that are well known to those of ordinary skill in the art, such as those described in U.S. Pat. Nos. 3,845,770; 3,916,899; 3,536,809; 3,598,123; 4,008,719; 4,710,384; 5,674,533; 5,059,595; 5,591,767; 5,120,548; 5,073,543; 5,639,476; 5,354,556; and 5,733,566, the disclosures of which are each incorporated herein by reference. The pharmaceutical compositions of the present invention can be used to provide slow or sustained release of one or more of the active ingredients using, for example, hydropropylmethyl cellulose, other polymer matrices, gels, permeable membranes, osmotic systems, multilayer coatings, microparticles, liposomes, microspheres, or the like, or a combination thereof to provide the desired release profile in varying proportions. Suitable sustained release formulations known to those of ordinary skill in the art, including those described herein, may be readily selected for use with the pharmaceutical compositions of the invention. Thus, single unit dosage forms suitable for oral administration, such as, but not limited to, tablets, capsules, gelcaps, caplets, powders, and the like, that are adapted for sustained release are encompassed by the present invention.

The dosage administered will be a therapeutically effective amount of the compound sufficient to result in amelioration of symptoms of the bone disease and will, of course, vary depending upon known factors such as the pharmacodynamic characteristics of the particular active ingredient and its mode and route of administration; age, sex, health and weight of the recipient; nature and extent of symptoms; kind of concurrent treatment, frequency of treatment and the effect desired.

Toxicity and therapeutic efficacy of therapeutic compositions of the present invention can be determined by standard pharmaceutical procedures in cell cultures or experimental animals, e.g., for determining The LD50 (The Dose Lethal To 50% Of The Population) and The ED50 (the dose therapeutically effective in 50% of the population). The dose ratio between toxic and therapeutic effects is the therapeutic index and it can be expressed as the ratio LD50/ED50. Therapeutic agents which exhibit large therapeutic induces are preferred. While therapeutic compositions that exhibit toxic side effects may be used, care should be taken to design a delivery system that targets such therapeutic agents to the site of affected tissue in order to minimize potential damage to uninfected cells and, thereby, reduce side effects.

The data obtained from cell culture assays and animal studies can be used in formulating a range of dosage for use in humans. The dosage lies preferably within a range of circulating concentrations that include the ED50 with little or no toxicity. The dosage may vary within this range depending upon the dosage form employed and the route of administration utilized. For any agents used in the method of the invention, the therapeutically effective dose can be estimated initially from cell culture assays. A dose may be formulated in animal models to achieve a circulating plasma concentration range that includes the IC50 (i.e., the concentration of the test therapeutic agent which achieves a half-maximal inhibition of symptoms or inhibition of biochemical activity) as determined in cell culture. Such information can be used to more accurately determine useful doses in humans. Levels in plasma may be measured, for example, by high performance liquid chromatography.

It is understood that appropriate doses of small molecule agents depends upon a number of factors known to those or ordinary skill in the art, e.g., a physician. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram.

These methods described herein are by no means all-inclusive, and further methods to suit the specific application will be apparent to the ordinary skilled artisan. Moreover, the effective amount of the compositions can be further approximated through analogy to compounds known to exert the desired effect.

The practice of aspects of the present invention may employ, unless otherwise indicated, conventional techniques of cell biology, cell culture, molecular biology, transgenic biology, microbiology, recombinant DNA, and immunology, which are within the skill of the art.

### EXAMPLES

The following examples are included to demonstrate embodiments. It should be appreciated by those skilled in the art that the techniques disclosed in the examples which follow represent techniques discovered by the inventors to function well in practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention.

### A. General Methods

Generally, nomenclatures utilized in connection with, and techniques of, cell and tissue culture, molecular biology, and protein and oligo- or polynucleotide chemistry and hybridization described herein are those well known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques of described herein are those well known and commonly used in the art.

Cell lines. The human HCC cell lines HepG2, PLC/PRF-5, Huh-7, SNU-182, SNU-449, and SK-Hep-1 were obtained from the American Type Culture Collection (Manassas, Va.). Normal human hepatocytes were obtained from Sciencell (San Diego, Calif.). Mouse hepatocyte cell lines BNL-CL.2 and BNL-SVA.8 (SV40-transformed BNL-CL.2 cells) were obtained from the American Type Culture Collection.

ucRNA expression profiling. RNA was extracted from three separate biological samples for each analysis using Trizol reagent (Invitrogen, Carlsbad, Calif.). Total RNA (5 μg) was reverse transcribed using biotin end-labeled random oligonucleotide primers and cDNA was hybridized to a custom microarray (OSU-CCC 4.0), which includes sense and antisense probes to all 481 human ultraconserved regions (UCRs), each spotted in duplicate. The chromosomal coordinate ranges for all 481 UCRs are listed below in table 3:

TABLE 3

| Name | Length | Build 34 (hg16) coords |
|---|---|---|
| uc.1 | 207 | chr1: 10307243-10307449 |
| uc.2 | 207 | chr1: 10442089-10442295 |
| uc.3 | 225 | chr1: 10460711-10460935 |
| uc.4 | 359 | chr1: 10467795-10468153 |
| uc.5 | 214 | chr1: 10490897-10491110 |
| uc.6 | 301 | chr1: 10504667-10504967 |
| uc.7 | 256 | chr1: 10545679-10545934 |
| uc.8 | 216 | chr1: 10561364-10561579 |
| uc.9 | 202 | chr1: 10634956-10635157 |
| uc.10 | 275 | chr1: 10675120-10675394 |
| uc.12 | 201 | chr1: 35077889-35078089 |
| uc.13 | 237 | chr1: 35786852-35787088 |
| uc.14 | 213 | chr1: 37908298-37908510 |
| uc.15 | 233 | chr1: 37974370-37974602 |
| uc.16 | 211 | chr1: 38041493-38041703 |
| uc.17 | 237 | chr1: 38215445-38215681 |
| uc.18 | 238 | chr1: 44128955-44129192 |
| uc.19 | 256 | chr1: 44403606-44403861 |
| uc.20 | 269 | chr1: 44415666-44415934 |
| uc.21 | 235 | chr1: 48482903-48483137 |
| uc.22 | 308 | chr1: 50376149-50376456 |
| uc.23 | 229 | chr1: 50405695-50405923 |
| uc.24 | 336 | chr1: 50469063-50469398 |
| uc.25 | 235 | chr1: 50535952-50536186 |
| uc.26 | 212 | chr1: 62739563-62739774 |
| uc.27 | 290 | chr1: 62739797-62740086 |
| uc.28 | 355 | chr1: 70066629-70066983 |
| uc.29 | 219 | chr1: 87244976-87245194 |
| uc.30 | 243 | chr1: 87451593-87451835 |
| uc.31 | 253 | chr1: 88395168-88395420 |
| uc.33 | 312 | chr1: 96743538-96743849 |
| uc.34 | 208 | chr1: 96751973-96752180 |
| uc.35 | 205 | chr1: 97465205-97465409 |
| uc.36 | 264 | chr1: 108587040-108587303 |
| uc.37 | 202 | chr1: 114578780-114578981 |
| uc.38 | 224 | chr1: 161127332-161127555 |
| uc.39 | 356 | chr1: 161210912-161211267 |
| uc.40 | 247 | chr1: 161825339-161825585 |
| uc.41 | 216 | chr1: 210655265-210655480 |
| uc.42 | 266 | chr1: 212945530-212945795 |
| uc.43 | 257 | chr1: 240842759-240843015 |
| uc.44 | 230 | chr1: 241164431-241164660 |
| uc.45 | 203 | chr1: 241963410-241963612 |
| uc.46 | 217 | chr1: 241964327-241964543 |
| uc.47 | 227 | chr2: 7796390-7796616 |
| uc.48 | 298 | chr2: 20462845-20463142 |
| uc.49 | 207 | chr2: 33787944-33788150 |
| uc.50 | 222 | chr2: 38950836-38951057 |
| uc.51 | 207 | chr2: 57947093-57947299 |
| uc.52 | 274 | chr2: 59082720-59082993 |
| uc.53 | 232 | chr2: 59107845-59108076 |
| uc.54 | 209 | chr2: 59173937-59174145 |
| uc.55 | 240 | chr2: 59721112-59721351 |
| uc.56 | 202 | chr2: 59922365-59922566 |
| uc.57 | 246 | chr2: 60113774-60114019 |
| uc.58 | 203 | chr2: 60272497-60272699 |
| uc.59 | 220 | chr2: 60272917-60273136 |
| uc.60 | 217 | chr2: 60416094-60416310 |
| uc.61 | 326 | chr2: 60662107-60662432 |
| uc.62 | 234 | chr2: 60755216-60755449 |
| uc.63 | 278 | chr2: 61727035-61727312 |
| uc.64 | 245 | chr2: 63168625-63168869 |
| uc.65 | 212 | chr2: 66273125-66273336 |
| uc.66 | 247 | chr2: 73149541-73149787 |
| uc.67 | 217 | chr2: 104358126-104358342 |
| uc.68 | 255 | chr2: 144125491-144125745 |
| uc.69 | 301 | chr2: 144322879-144323179 |
| uc.70 | 237 | chr2: 144648108-144648344 |
| uc.71 | 248 | chr2: 144923625-144923872 |
| uc.72 | 407 | chr2: 144925741-144926147 |
| uc.73 | 201 | chr2: 144973082-144973282 |
| uc.74 | 538 | chr2: 145036732-145037269 |
| uc.75 | 236 | chr2: 145356619-145356854 |
| uc.76 | 335 | chr2: 145371902-145372236 |
| uc.77 | 296 | chr2: 145396534-145396829 |
| uc.78 | 248 | chr2: 145399123-145399370 |
| uc.79 | 295 | chr2: 145407946-145408240 |
| uc.80 | 294 | chr2: 145411621-145411914 |
| uc.81 | 211 | chr2: 147344834-147345044 |
| uc.82 | 210 | chr2: 156929644-156929853 |
| uc.83 | 296 | chr2: 157194172-157194467 |
| uc.84 | 209 | chr2: 157397251-157397459 |
| uc.85 | 248 | chr2: 157753959-157754206 |
| uc.86 | 340 | chr2: 157862655-157862994 |
| uc.87 | 290 | chr2: 158102814-158103103 |
| uc.88 | 312 | chr2: 162297586-162297897 |
| uc.89 | 307 | chr2: 162441217-162441523 |
| uc.90 | 206 | chr2: 162475571-162475776 |
| uc.91 | 207 | chr2: 163247566-163247772 |
| uc.92 | 309 | chr2: 164653223-164653531 |
| uc.93 | 263 | chr2: 164864451-164864713 |
| uc.94 | 200 | chr2: 165046714-165046913 |
| uc.95 | 251 | chr2: 171774074-171774324 |
| uc.96 | 261 | chr2: 173023218-173023478 |
| uc.97 | 442 | chr2: 173025175-173025616 |
| uc.98 | 238 | chr2: 173159062-173159299 |
| uc.99 | 398 | chr2: 173160925-173161322 |
| uc.100 | 207 | chr2: 174317324-174317530 |
| uc.101 | 254 | chr2: 174977025-174977278 |
| uc.102 | 338 | chr2: 175148953-175149290 |
| uc.103 | 233 | chr2: 175172216-175172448 |
| uc.104 | 216 | chr2: 175189478-175189693 |
| uc.105 | 223 | chr2: 175192331-175192553 |
| uc.106 | 206 | chr2: 175227967-175228172 |
| uc.107 | 237 | chr2: 175410152-175410388 |
| uc.108 | 374 | chr2: 177142901-177143274 |
| uc.109 | 224 | chr2: 177705882-177706105 |
| uc.110 | 243 | chr2: 237358132-237358374 |
| uc.111 | 296 | chr3: 9446461-9446756 |
| uc.483 | 402 | chr3: 17567733-17568134 |
| uc.112 | 346 | chr3: 18144568-18144913 |
| uc.113 | 247 | chr3: 18651408-18651654 |
| uc.114 | 294 | chr3: 18819454-18819747 |
| uc.115 | 219 | chr3: 19009162-19009380 |
| uc.116 | 206 | chr3: 70499494-70499699 |
| uc.117 | 251 | chr3: 70792683-70792933 |
| uc.118 | 219 | chr3: 70792935-70793153 |
| uc.119 | 301 | chr3: 115754368-115754668 |
| uc.120 | 270 | chr3: 115755940-115756209 |
| uc.121 | 293 | chr3: 115896375-115896667 |
| uc.122 | 215 | chr3: 115932792-115933006 |
| uc.123 | 492 | chr3: 138304453-138304944 |
| uc.124 | 287 | chr3: 138369353-138369639 |
| uc.125 | 265 | chr3: 138389226-138389490 |
| uc.126 | 271 | chr3: 138446557-138446827 |
| uc.127 | 272 | chr3: 148351617-148351888 |
| uc.128 | 299 | chr3: 148370547-148370845 |
| uc.129 | 212 | chr3: 153485296-153485507 |
| uc.130 | 224 | chr3: 159097487-159097710 |
| uc.131 | 207 | chr3: 159310953-159311159 |
| uc.132 | 208 | chr3: 159347072-159347279 |
| uc.133 | 277 | chr3: 159347391-159347667 |
| uc.134 | 211 | chr3: 159566817-159567027 |
| uc.135 | 201 | chr3: 170155196-170155396 |
| uc.136 | 347 | chr3: 170514865-170515211 |
| uc.137 | 385 | chr3: 181757770-181758154 |
| uc.138 | 419 | chr3: 186970209-186970627 |
| uc.139 | 338 | chr4: 4587982-4588319 |
| uc.140 | 223 | chr4: 12760753-12760975 |
| uc.141 | 295 | chr4: 24280045-24280339 |
| uc.142 | 259 | chr4: 41665611-41665869 |
| uc.143 | 218 | chr4: 77037519-77037736 |
| uc.144 | 205 | chr4: 83805060-83805264 |
| uc.145 | 248 | chr4: 105805133-105805380 |
| uc.146 | 214 | chr4: 112375296-112375509 |
| uc.147 | 308 | chr4: 151814010-151814317 |
| uc.148 | 240 | chr4: 152071579-152071818 |
| uc.149 | 204 | chr4: 152071820-152072023 |
| uc.150 | 262 | chr5: 3565359-3565620 |
| uc.151 | 214 | chr5: 32425638-32425851 |
| uc.152 | 201 | chr5: 50351523-50351723 |
| uc.153 | 240 | chr5: 72279759-72279998 |
| uc.154 | 203 | chr5: 72294088-72294290 |
| uc.155 | 207 | chr5: 77018437-77018643 |
| uc.156 | 213 | chr5: 77019492-77019704 |
| uc.157 | 207 | chr5: 77025234-77025440 |
| uc.158 | 224 | chr5: 77224326-77224549 |
| uc.159 | 472 | chr5: 77232005-77232476 |
| uc.160 | 322 | chr5: 77352917-77353238 |
| uc.161 | 278 | chr5: 77442602-77442879 |
| uc.162 | 218 | chr5: 81231434-81231651 |
| uc.163 | 376 | chr5: 87252696-87253071 |
| uc.164 | 203 | chr5: 87324478-87324680 |
| uc.165 | 224 | chr5: 87777006-87777229 |
| uc.166 | 310 | chr5: 88045878-88046187 |
| uc.167 | 201 | chr5: 88263697-88263897 |
| uc.168 | 220 | chr5: 91012866-91013085 |
| uc.169 | 204 | chr5: 92995090-92995293 |
| uc.170 | 310 | chr5: 93301743-93302052 |
| uc.171 | 208 | chr5: 93649920-93650127 |
| uc.172 | 218 | chr5: 93724792-93725009 |
| uc.173 | 276 | chr5: 133802376-133802651 |
| uc.174 | 260 | chr5: 138719870-138720129 |
| uc.175 | 250 | chr5: 158322733-158322982 |
| uc.176 | 246 | chr5: 167313590-167313835 |
| uc.177 | 257 | chr5: 170398551-170398807 |
| uc.178 | 249 | chr5: 170398920-170399168 |
| uc.179 | 219 | chr5: 170609134-170609352 |
| uc.180 | 225 | chr5: 170609411-170609635 |
| uc.181 | 278 | chr5: 170610401-170610678 |
| uc.182 | 239 | chr5: 170684001-170684239 |
| uc.183 | 236 | chr5: 171365442-171365677 |
| uc.184 | 230 | chr5: 173366215-173366444 |
| uc.185 | 411 | chr5: 178157908-178158318 |
| uc.186 | 305 | chr5: 179155889-179156193 |
| uc.187 | 212 | chr6: 10502663-10502874 |
| uc.188 | 215 | chr6: 16407363-16407577 |
| uc.189 | 573 | chr6: 36614372-36614944 |
| uc.190 | 200 | chr6: 41570295-41570494 |
| uc.191 | 208 | chr6: 51123630-51123837 |
| uc.192 | 244 | chr6: 51195833-51196076 |
| uc.193 | 319 | chr6: 86317282-86317600 |
| uc.194 | 201 | chr6: 93964662-93964862 |
| uc.195 | 273 | chr6: 97708954-97709226 |
| uc.196 | 221 | chr6: 98162138-98162358 |
| uc.197 | 224 | chr6: 98408397-98408620 |
| uc.198 | 307 | chr6: 98765487-98765793 |
| uc.199 | 256 | chr6: 98859453-98859708 |
| uc.200 | 254 | chr6: 99041131-99041384 |
| uc.201 | 240 | chr6: 100097582-100097821 |
| uc.202 | 230 | chr6: 101019581-101019810 |
| uc.203 | 203 | chr6: 163900992-163901194 |
| uc.204 | 202 | chr7: 1010242-1010443 |
| uc.205 | 252 | chr7: 20574105-20574356 |
| uc.206 | 499 | chr7: 20748081-20748579 |
| uc.207 | 230 | chr7: 21555809-21556038 |
| uc.208 | 218 | chr7: 23303942-23304159 |
| uc.209 | 250 | chr7: 23304160-23304409 |
| uc.210 | 257 | chr7: 26439350-26439606 |
| uc.211 | 291 | chr7: 26471744-26472034 |
| uc.212 | 205 | chr7: 26884210-26884414 |
| uc.213 | 201 | chr7: 26925404-26925604 |
| uc.214 | 243 | chr7: 31144670-31144912 |
| uc.215 | 262 | chr7: 41933365-41933626 |
| uc.216 | 312 | chr7: 50102955-50103266 |
| uc.217 | 221 | chr7: 54378405-54378625 |
| uc.218 | 286 | chr7: 69215092-69215377 |
| uc.219 | 210 | chr7: 69392897-69393106 |
| uc.220 | 257 | chr7: 96245647-96245903 |
| uc.221 | 349 | chr7: 96253032-96253380 |
| uc.222 | 201 | chr7: 113611674-113611874 |
| uc.223 | 268 | chr7: 113612688-113612955 |
| uc.224 | 295 | chr7: 113617522-113617816 |
| uc.225 | 201 | chr7: 113627358-113627558 |
| uc.226 | 205 | chr7: 113763821-113764025 |
| uc.227 | 231 | chr7: 113849819-113850049 |
| uc.228 | 265 | chr7: 114671200-114671464 |
| uc.229 | 296 | chr7: 114689148-114689443 |
| uc.230 | 238 | chr7: 114873964-114874201 |
| uc.231 | 224 | chr7: 115136620-115136843 |
| uc.232 | 247 | chr7: 121499109-121499355 |
| uc.233 | 266 | chr7: 150220055-150220320 |
| uc.234 | 272 | chr7: 156229240-156229511 |
| uc.235 | 227 | chr8: 25797831-25798057 |
| uc.236 | 267 | chr8: 37307753-37308019 |
| uc.237 | 468 | chr8: 53187862-53188329 |
| uc.238 | 358 | chr8: 53217077-53217434 |
| uc.239 | 300 | chr8: 59992334-59992633 |
| uc.240 | 206 | chr8: 65542500-65542705 |
| uc.241 | 202 | chr8: 65547091-65547292 |
| uc.242 | 265 | chr8: 66199258-66199522 |
| uc.243 | 216 | chr8: 77740924-77741139 |
| uc.244 | 321 | chr8: 105918932-105919252 |
| uc.245 | 339 | chr8: 106290415-106290753 |
| uc.246 | 284 | chr8: 119079806-119080089 |
| uc.247 | 361 | chr9: 959154-959514 |
| uc.248 | 222 | chr9: 964189-964410 |
| uc.249 | 260 | chr9: 8085728-8085987 |
| uc.250 | 209 | chr9: 13929910-13930118 |
| uc.251 | 213 | chr9: 15864309-15864521 |
| uc.252 | 231 | chr9: 16700753-16700983 |
| uc.253 | 222 | chr9: 17322212-17322433 |
| uc.254 | 279 | chr9: 23486725-23487003 |
| uc.255 | 232 | chr9: 23681768-23681999 |
| uc.256 | 206 | chr9: 23682234-23682439 |
| uc.257 | 264 | chr9: 37205204-37205467 |
| uc.258 | 201 | chr9: 37314424-37314624 |
| uc.259 | 308 | chr9: 75084998-75085305 |
| uc.260 | 231 | chr9: 76929543-76929773 |
| uc.261 | 311 | chr9: 77328693-77329003 |
| uc.262 | 255 | chr9: 79184841-79185095 |
| uc.263 | 207 | chr9: 82047403-82047609 |
| uc.264 | 267 | chr9: 82047611-82047877 |
| uc.265 | 217 | chr9: 103498309-103498525 |
| uc.266 | 243 | chr9: 104758130-104758372 |
| uc.267 | 203 | chr9: 120429935-120430137 |
| uc.268 | 251 | chr9: 120982873-120983123 |
| uc.269 | 217 | chr9: 121913982-121914198 |
| uc.270 | 278 | chr9: 123680114-123680391 |
| uc.271 | 211 | chr9: 123680397-123680607 |
| uc.272 | 213 | chr9: 123808633-123808845 |
| uc.273 | 321 | chr9: 123893643-123893963 |
| uc.274 | 327 | chr9: 123897916-123898242 |
| uc.275 | 255 | chr9: 123960161-123960415 |
| uc.276 | 432 | chr9: 123981857-123982288 |
| uc.277 | 276 | chr9: 123983755-123984030 |
| uc.278 | 237 | chr9: 124022210-124022446 |
| uc.279 | 336 | chr9: 124048604-124048939 |
| uc.280 | 220 | chr9: 124054051-124054270 |
| uc.281 | 238 | chr9: 130771570-130771807 |
| uc.282 | 207 | chr9: 135399783-135399989 |
| uc.283 | 277 | chr10: 49949360-49949636 |
| uc.284 | 209 | chr10: 49951459-49951667 |
| uc.285 | 232 | chr10: 69860594-69860825 |
| uc.286 | 252 | chr10: 76483627-76483878 |
| uc.287 | 257 | chr10: 76840546-76840802 |
| uc.288 | 223 | chr10: 77071786-77072008 |
| uc.289 | 254 | chr10: 77335142-77335395 |
| uc.290 | 206 | chr10: 77386885-77387090 |
| uc.291 | 231 | chr10: 77628227-77628457 |
| uc.292 | 217 | chr10: 98380042-98380258 |
| uc.293 | 243 | chr10: 102037256-102037498 |
| uc.294 | 444 | chr10: 102038204-102038647 |
| uc.295 | 209 | chr10: 102039687-102039895 |
| uc.296 | 461 | chr10: 102079693-102080153 |
| uc.297 | 364 | chr10: 102083807-102084170 |
| uc.298 | 359 | chr10: 102112245-102112603 |
| uc.299 | 210 | chr10: 102174022-102174231 |
| uc.300 | 208 | chr10: 102211705-102211912 |
| uc.301 | 284 | chr10: 102232378-102232661 |
| uc.302 | 341 | chr10: 102643767-102644107 |
| uc.303 | 272 | chr10: 102717014-102717285 |
| uc.304 | 272 | chr10: 102747091-102747362 |
| uc.305 | 305 | chr10: 102876022-102876326 |
| uc.306 | 224 | chr10: 102876626-102876849 |
| uc.307 | 232 | chr10: 102908570-102908801 |
| uc.308 | 277 | chr10: 102910399-102910675 |
| uc.309 | 268 | chr10: 102931618-102931885 |
| uc.310 | 229 | chr10: 114068810-114069038 |
| uc.311 | 219 | chr10: 119738989-119739207 |
| uc.312 | 322 | chr10: 119741124-119741445 |
| uc.313 | 231 | chr10: 121004761-121004991 |
| uc.314 | 202 | chr10: 124392535-124392736 |
| uc.315 | 235 | chr10: 124392947-124393181 |
| uc.316 | 240 | chr10: 126479965-126480204 |
| uc.317 | 218 | chr10: 130920738-130920955 |
| uc.318 | 321 | chr10: 131165986-131166306 |
| uc.319 | 316 | chr11: 8269004-8269319 |
| uc.320 | 335 | chr11: 8282143-8282477 |
| uc.321 | 204 | chr11: 15588733-15588936 |
| uc.322 | 223 | chr11: 16280660-16280882 |
| uc.323 | 200 | chr11: 16439645-16439844 |
| uc.324 | 225 | chr11: 30521830-30522054 |
| uc.325 | 235 | chr11: 31649953-31650187 |
| uc.326 | 315 | chr11: 31749989-31750303 |
| uc.327 | 268 | chr11: 31750593-31750860 |
| uc.328 | 231 | chr11: 31789972-31790202 |
| uc.329 | 307 | chr11: 32162301-32162607 |
| uc.330 | 207 | chr11: 66169256-66169462 |
| uc.331 | 218 | chr11: 82921467-82921684 |
| uc.332 | 337 | chr11: 115770342-115770678 |
| uc.333 | 270 | chr11: 124182299-124182568 |
| uc.334 | 222 | chr11: 131405597-131405818 |
| uc.335 | 214 | chr12: 16606681-16606894 |
| uc.336 | 251 | chr12: 24183273-24183523 |
| uc.337 | 218 | chr12: 40035446-40035663 |
| uc.338 | 223 | chr12: 52144756-52144978 |
| uc.339 | 252 | chr12: 52357363-52357614 |
| uc.340 | 259 | chr12: 52377099-52377357 |
| uc.341 | 314 | chr12: 52669185-52669498 |
| uc.342 | 227 | chr12: 52696761-52696987 |
| uc.343 | 388 | chr12: 52708708-52709095 |
| uc.344 | 254 | chr12: 52713153-52713406 |
| uc.345 | 301 | chr12: 52733867-52734167 |
| uc.346 | 202 | chr12: 105478977-105479178 |
| uc.347 | 209 | chr13: 69591989-69592197 |
| uc.348 | 240 | chr13: 69861358-69861597 |
| uc.349 | 203 | chr13: 69919303-69919505 |
| uc.350 | 240 | chr13: 70054101-70054340 |
| uc.351 | 255 | chr13: 70466901-70467155 |
| uc.352 | 200 | chr13: 70492166-70492365 |
| uc.353 | 323 | chr13: 70569554-70569876 |
| uc.354 | 235 | chr13: 76774830-76775064 |
| uc.355 | 228 | chr13: 93316883-93317110 |
| uc.356 | 251 | chr13: 95706821-95707071 |
| uc.357 | 242 | chr13: 110664338-110664579 |
| uc.358 | 226 | chr14: 24368162-24368387 |
| uc.359 | 324 | chr14: 24905096-24905419 |
| uc.360 | 287 | chr14: 24905510-24905796 |
| uc.361 | 267 | chr14: 27223174-27223440 |
| uc.362 | 239 | chr14: 27338791-27339029 |
| uc.363 | 265 | chr14: 27851358-27851622 |
| uc.364 | 207 | chr14: 28702798-28703004 |
| uc.365 | 278 | chr14: 28732401-28732678 |
| uc.366 | 202 | chr14: 29372746-29372947 |
| uc.367 | 298 | chr14: 31834502-31834799 |
| uc.368 | 228 | chr14: 32058615-32058842 |
| uc.369 | 213 | chr14: 32112656-32112868 |
| uc.370 | 393 | chr14: 32192610-32193002 |
| uc.371 | 296 | chr14: 34010228-34010523 |
| uc.372 | 277 | chr14: 34033074-34033350 |
| uc.373 | 394 | chr14: 34571846-34572239 |
| uc.374 | 224 | chr14: 35705952-35706175 |
| uc.375 | 300 | chr14: 35767259-35767558 |
| uc.376 | 290 | chr14: 43555788-43556077 |
| uc.377 | 217 | chr14: 43569061-43569277 |
| uc.378 | 251 | chr14: 78317518-78317768 |
| uc.379 | 252 | chr14: 95421409-95421660 |
| uc.380 | 232 | chr14: 95752635-95752866 |
| uc.381 | 238 | chr14: 95869331-95869568 |
| uc.382 | 200 | chr15: 33634968-33635167 |
| uc.383 | 269 | chr15: 34535983-34536251 |
| uc.384 | 266 | chr15: 34681449-34681714 |
| uc.385 | 209 | chr15: 34901726-34901934 |
| uc.386 | 203 | chr15: 35238065-35238267 |
| uc.387 | 238 | chr15: 39748110-39748347 |
| uc.388 | 298 | chr15: 55141581-55141878 |
| uc.389 | 271 | chr15: 65381203-65381473 |
| uc.390 | 205 | chr15: 65593988-65594192 |
| uc.391 | 311 | chr15: 65756122-65756432 |
| uc.392 | 256 | chr15: 68107952-68108207 |
| uc.393 | 275 | chr15: 72630059-72630333 |
| uc.394 | 202 | chr15: 94965868-94966069 |
| uc.395 | 249 | chr16: 24545554-24545802 |
| uc.396 | 208 | chr16: 48872692-48872899 |
| uc.397 | 311 | chr16: 49513876-49514186 |
| uc.398 | 322 | chr16: 49668919-49669240 |
| uc.399 | 214 | chr16: 50627024-50627237 |
| uc.400 | 206 | chr16: 51450088-51450293 |
| uc.401 | 250 | chr16: 52273300-52273549 |
| uc.402 | 245 | chr16: 53432987-53433231 |
| uc.403 | 206 | chr16: 54102457-54102662 |
| uc.404 | 235 | chr16: 55001959-55002193 |
| uc.405 | 210 | chr16: 59350792-59351001 |
| uc.406 | 211 | chr16: 69456552-69456762 |
| uc.407 | 326 | chr16: 69457343-69457668 |
| uc.408 | 252 | chr16: 72597321-72597572 |
| uc.409 | 244 | chr16: 72869147-72869390 |
| uc.410 | 219 | chr17: 35207780-35207998 |
| uc.411 | 229 | chr17: 35525169-35525397 |
| uc.412 | 268 | chr17: 35532036-35532303 |
| uc.413 | 272 | chr17: 37941482-37941753 |
| uc.414 | 246 | chr17: 38624137-38624382 |
| uc.415 | 207 | chr17: 47138544-47138750 |
| uc.416 | 286 | chr17: 47145525-47145810 |
| uc.417 | 222 | chr17: 47156950-47157171 |
| uc.418 | 217 | chr17: 56556866-56557082 |
| uc.419 | 289 | chr17: 56557353-56557641 |
| uc.420 | 233 | chr17: 63046872-63047104 |
| uc.421 | 345 | chr18: 20945142-20945486 |
| uc.422 | 226 | chr18: 21000175-21000400 |
| uc.423 | 223 | chr18: 21008342-21008564 |
| uc.424 | 215 | chr18: 21019766-21019980 |
| uc.425 | 325 | chr18: 21117194-21117518 |
| uc.426 | 262 | chr18: 22167579-22167840 |
| uc.427 | 215 | chr18: 22489186-22489400 |
| uc.428 | 239 | chr18: 28605196-28605434 |
| uc.429 | 238 | chr18: 32732528-32732765 |
| uc.430 | 213 | chr18: 33315637-33315849 |
| uc.431 | 230 | chr18: 33430587-33430816 |
| uc.432 | 211 | chr18: 33816919-33817129 |
| uc.433 | 206 | chr18: 34315619-34315824 |
| uc.434 | 249 | chr18: 43022775-43023023 |
| uc.435 | 227 | chr18: 51238918-51239144 |
| uc.436 | 210 | chr18: 51403228-51403437 |
| uc.437 | 215 | chr18: 70484635-70484849 |
| uc.438 | 241 | chr18: 70484851-70485091 |
| uc.439 | 263 | chr18: 70490008-70490270 |
| uc.440 | 329 | chr18: 70490342-70490670 |
| uc.441 | 248 | chr18: 70695569-70695816 |
| uc.442 | 249 | chr18: 70719689-70719937 |
| uc.443 | 239 | chr19: 8433269-8433507 |
| uc.444 | 388 | chr19: 35186619-35187006 |
| uc.445 | 310 | chr19: 35258275-35258584 |
| uc.446 | 272 | chr19: 35439694-35439965 |
| uc.447 | 273 | chr19: 35459621-35459893 |
| uc.448 | 232 | chr19: 35533370-35533601 |
| uc.449 | 290 | chr19: 35695381-35695670 |
| uc.450 | 211 | chr19: 36279466-36279676 |
| uc.451 | 225 | chr19: 36498460-36498684 |
| uc.452 | 204 | chr19: 36519787-36519990 |
| uc.453 | 325 | chr19: 47129157-47129481 |
| uc.454 | 208 | chr20: 4861440-4861647 |
| uc.455 | 245 | chr20: 35043808-35044052 |
| uc.456 | 320 | chr20: 42773185-42773504 |
| uc.457 | 211 | chr22: 17770463-17770673 |
| uc.458 | 204 | chr22: 34420305-34420508 |
| uc.459 | 255 | chrX: 20895986-20896240 |
| uc.460 | 275 | chrX: 24184937-24185211 |
| uc.461 | 397 | chrX: 24226223-24226619 |
| uc.462 | 779 | chrX: 24256252-24257030 |
| uc.463 | 275 | chrX: 24277308-24277582 |
| uc.464 | 770 | chrX: 24277584-24278353 |
| uc.465 | 310 | chrX: 24278907-24279216 |
| uc.466 | 349 | chrX: 24307884-24308232 |
| uc.467 | 731 | chrX: 24369780-24370510 |
| uc.468 | 489 | chrX: 24378989-24379477 |
| uc.469 | 222 | chrX: 24379479-24379700 |
| uc.470 | 341 | chrX: 24762642-24762982 |
| uc.471 | 239 | chrX: 40239309-40239547 |
| uc.472 | 202 | chrX: 40410244-40410445 |
| uc.473 | 222 | chrX: 69240015-69240236 |
| uc.474 | 210 | chrX: 69335634-69335843 |
| uc.475 | 397 | chrX: 69632846-69633242 |
| uc.476 | 238 | chrX: 80545473-80545710 |
| uc.477 | 209 | chrX: 101813348-101813556 |
| uc.478 | 252 | chrX: 121297212-121297463 |
| uc.479 | 302 | chrX: 121311506-121311807 |
| uc.480 | 202 | chrX: 121933027-121933228 |
| uc.481 | 204 | chrX: 121933230-121933433 |

Sequence information for all 481 UCRs is available at http://genome.ucsc.edu/cgi-bin/hgGateway (July 2003 human reference sequence (NCBI Build 34) was produced by the International Human Genome Sequencing Consortium).

Biotin-containing transcripts were detected using streptavidin—Alexa647 conjugate, scanned and analyzed using an Axon 4000B scanner and the GenePix 6.0 software (Axon Instruments, Downingtown, Pa.). The mean fluorescence intensity of replicate spots were subtracted from background and normalized using the global median method. We selected ucRNAs measured as present in all the three replicates. Differentially expressed ucRNAs were identified using the Class Comparison Analysis of BRB tools version 3.6.0 (http://linus.nci.nih.gov/BRB-ArrayTools.html). The criterion for inclusion of a gene in the gene list was a p-value <0.05.

Real-time PCR analysis. RNA was extracted using Trizol reagent and treated with RNase-free DNase I (Qiagen, Valenica, Calif.). One μg of RNA was reverse transcribed to cDNA and quantitative real time PCR was performed using primers for uc.338 that spanned an intronic region, or primers for PCBP2 that spanned exonic regions distant to the location of uc.338, as follows: uc.338: 5′-AGCGACAGTGCGAGCTTT-3′, 3′-GGAAGGATTGAGTGAGCCTT-5′; PCBP2: 5′-TGACGCATGGCAACACC-3′, 5′-CGCCTTGACGCCCGATT-3′. Uc.338 and PCBP2 expression was normalized to that of RNU6B or GAPDH respectively. Genomic contamination was excluded by PCR of controls lacking reverse transcriptase for each sample.

Cell fractionation. Cells (2×10⁶) were pelleted and cytoplasmic and nuclear fractions obtained using the NE-PER Nuclear and Cytoplasmic Extraction Kit (Sigma, St Louis, Mo.). RNA from each fraction was extracted using Trizol, and purity assessed using an Agilent bioanalyzer to detect the transfer RNA electropherogram band only in the cytosolic fractions as previously described (2).

Transfection. Huh-7 cells were transfected using Lipofectamine 2000 reagent (Invitrogen, Carlsbad, Calif.) whereas HepG2 cells were transfected using the Nucleofector system, solution V program T28 (Amaxa Biosystems, Koln, Germany). siRNAs against uc.338 were designed using siDESIGN (http://www.dharmacon.com/sidesign), and the two highest ranked target sequences synthesized with sequences: siRNA-1 UGACAGCCCUGGAGACUGA and siRNA-2 CCACAGGACAGGUACAGCA. Cells were transfected with 100 nM siRNA to TUC338 or control (Dharmacon, Chicago, Ill.) for 48 hours before further experiments. siRNA against PCBP2 were purchased from Ambion (Ambion, Austin, Tex.).

RACE. HepG2 RNA was used to generate RACE-ready cDNA by using the SMARTer RACE cDNA Amplification Kit (Clontech, Mountainview, Calif.) and following manufacturer's protocol. cDNA ends were amplified with Universal Primer Mix and gene-specific primers. Placental RNA and transferring receptor-specific primers provided by the manufacturer were used as controls. In case the primary PCT failed to give distint bands, we performed a “nested” PCR with the nested universal primer and the nested gene-specific primers. Primers were as follows (5′ to 3′): SI (5′ RACE), CAGTCTCGGCTGAGGCGGAAGGATTG; ASI (5′ RACE), GTGCCGGATTGACAGCCCTGGAGAC; SE (5′ RACE), CTCTAGAGGTGGTCCCTCCAGGTCAGG; nSI (5′ RACE), AAGGCTCACTCAATCCTTCC; nested ASI, TGACAGCCCTGGAGACTGAAATCCT; and antisense exonic, GGACAGGTACAGCACAGGCAGCGACAG. For the 3′ RACE RNA was polyadenylated by using the poly(A) polymerase TAK2180 (Takara). PCR products were then run in a 1.5% agarose gel, and DNA was extracted, cloned in TOPO.TA.2 plasmid, and sequenced with a 48-capillary Applied Biosystems 3730 DNA Analyzer.

Western Blotting. HepG2 and Huh7 cells were serum-starved for 48 hours and then transfected as described above. After 48 hours, cells were collected and protein was extracted. Immunoblot analysis was performed as previously described (X). The primary anti-bodies were used at a concentration of 1:500 and were as follows: rabbit polyclonal anti-p16-INK4, mouse monoclonal anti-CDK4, and mouse monoclonal CDK6 (Cell Signaling); mouse monoclonal anti-cyclin D1 (BD Biosciences); and mouse monoclonal anti-proliferating cell nuclear antigen and rabbit polyclonal anti-vinculin (Santa Cruz Biotechnology, Santa Cruz, Calif.).

Northern Blotting. For uc.338 detection, polyacrylamide gel-based Northern blotting was performed as previously described (4). The oligonucleotide probes used were complementary to the entire sequence of TUC338 or the entire cDNA of PCBP2. The size of the detected RNA was determined by using a size marker run on the same gel.

uc.338 cloning. Genomic DNA was extracted from HepG2 cells using DNAzol (Invitrogen, Carlsbad, Calif.). The entire sequence of uc.338 was amplified using the following primers: uc.338-F: 5′-TTTTGTGTGAATTTCATGCTGGT-3′ and uc.338-R: 5′-GGTGTCCACAGCACCAAAAA-3′. The PCR product was sub-cloned into TOPO TA2.1 cloning vector (Invitrogen Carlsbad, Calif.), digested with Hind-III and Not-I (New England Biolabs, Ipswich, Mass.) and then cloned into the pSUPER.retro.neo+gfp vector (OligoEngine Seattle Wash.). The final product was verified by sequencing. Cells were transfected with 5 μg of uc.338-expressing or empty vector using lipofectamine and used after 48 hours.

Gene annotation enrichment analysis. mRNA expression analysis was performed using Human Exon 1.0ST array (Affymetrix, Santa Clara, Calif.) in RNA obtained from HepG2 cells that were transfected with either control oligonucleotides or LNA-antisense to TUC338, which reduced uc.338 expression by 40% compared to controls by real-time PCR. Data were analyzed using the XRay software (Biotique Systems Inc, Reno, Nev.). Genes that were significantly differentially expressed were identified, and then compared to Gene Ontology classifications to identify over-representation in groups of the molecular function, cell processes or pathway classes. Microarray data has been deposited in the NCBI GEO repository.

Cell growth assays. For anchorage-dependent growth, cells were plated (2×10⁵/well) in 6-well plates. Trypan blue staining was performed at each time point and the number of viable cells was expressed relative to cell counts at baseline. For anchorage-independent cell growth, cells were plated in 96-well plates (1000/well) in 0.6% agar containing medium with 20% FBS with 0.8% agar containing top and bottom feeder layers. Cell growth was fluorometrically assayed after 7 days using Alamar Blue (Biosource International, Camarillo, Calif.), and a FluoStar Omega Microplate Reader (BMG Labtech, Durham, N.C.). Final values were obtained by subtracting background fluorescence values from wells without cells.

Cell cycle analysis. HepG2 cells were permeabilized with 75% ethanol and DNA stained using 50 μg/ml propidium iodide (PI), 0.1 mg/ml RNase A, 0.05% triton X-100 PBS. Cellular DNA content was measured by flow cytometry using a BD FACSCalibur (Heidelberg, Germany), and the proportions of cells in particular phases of the cell cycle were analyzed using the CellQuest Pro software.

Tissue Microarray (TMA). A tissue microarray (TMA) was constructed from paraffin embedded-tissue samples obtained from 191 patients with HCC and adjacent non-tumoral tissue as previously described. Sections from the TMA block were used for in situ hybridization analysis. A separate commercially available TMA with 30 cases of HCC spotted in duplicate was also analyzed (Accumax array, IsuAbxis, Seoul, Korea).

In situ RNA hybridization (ISH). A locked nucleic acid (LNA) probe with complementarity to a 22 bp section of uc.338 was labeled with 5′-digoxigenin and synthesized by Exiqon (Woburn, Mass., USA). Tissue sections on the TMA were digested using 2 mg/mL pepsin and ISH performed as described. Negative controls included omission of the probe and the use of a scrambled LNA probe. Each sample was classified by two independent reviewers based on the percentage of cells with detectable uc.338 expression as follows: negative (<5%), weak (5-19%), moderate (20-49%) or strong (≧50%). An expression score was derived as the difference in percentage of cells that expressed uc.338 in HCC and in the corresponding adjacent liver, divided by the SD of % uc.338 expression across all samples analyzed.

Statistical analysis. Results are expressed as mean±SEM, unless indicated otherwise. Comparisons between groups were performed using the two-tailed Student's t test. Significance was accepted when p was less than 0.05.

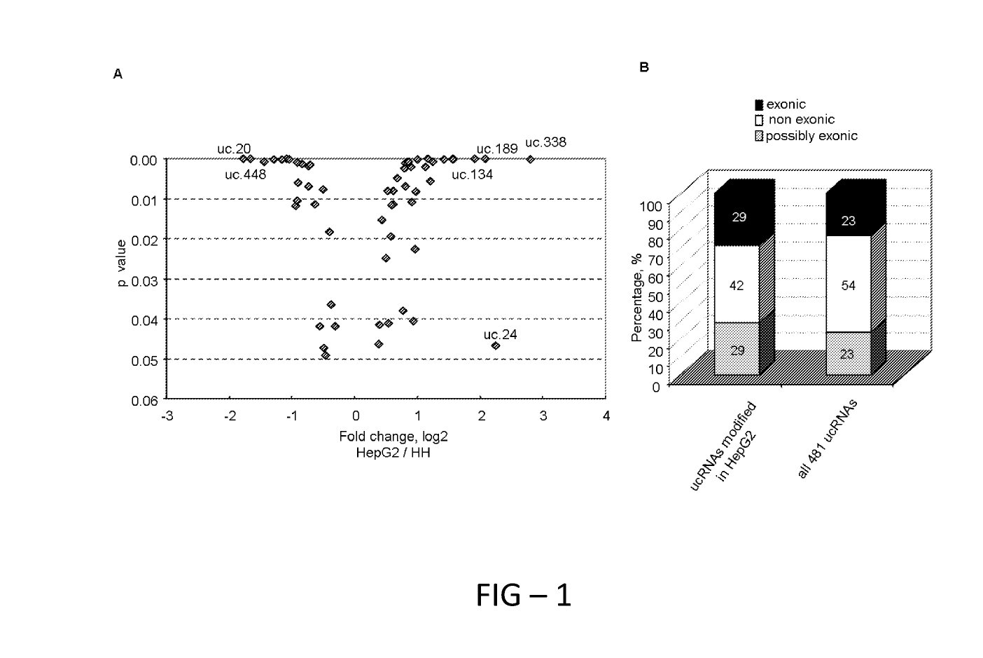

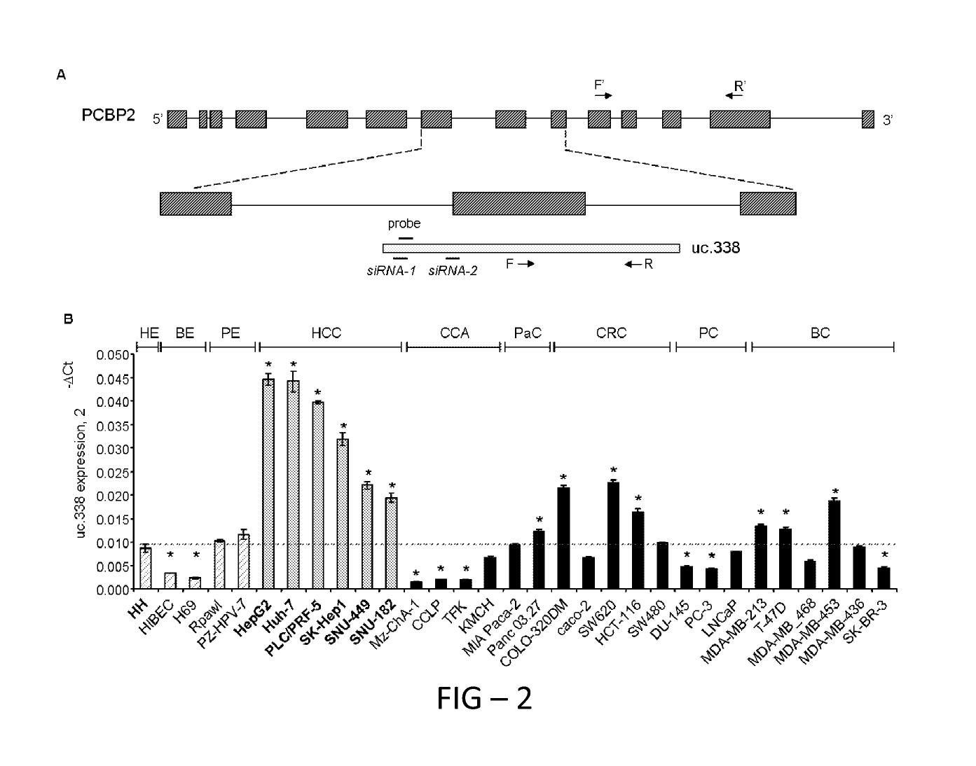

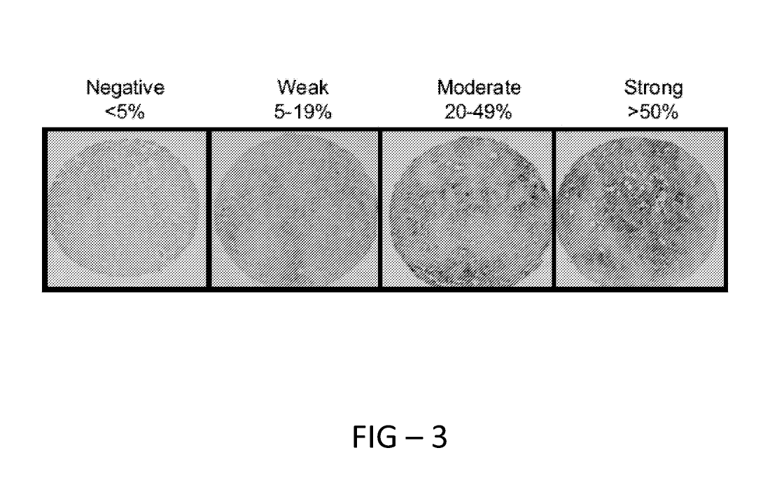

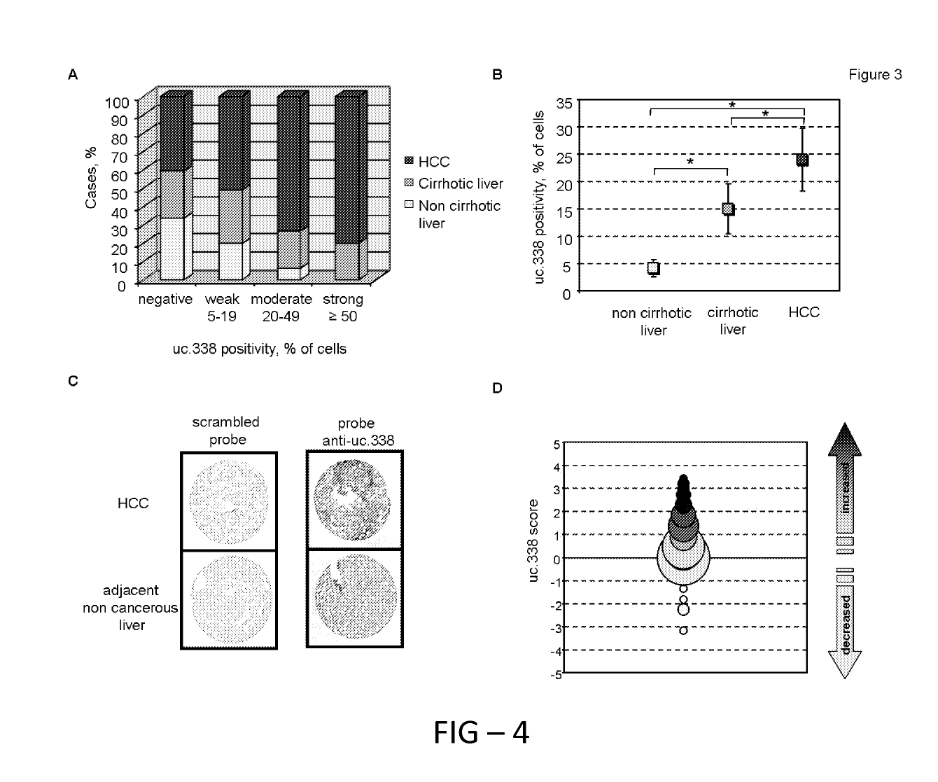

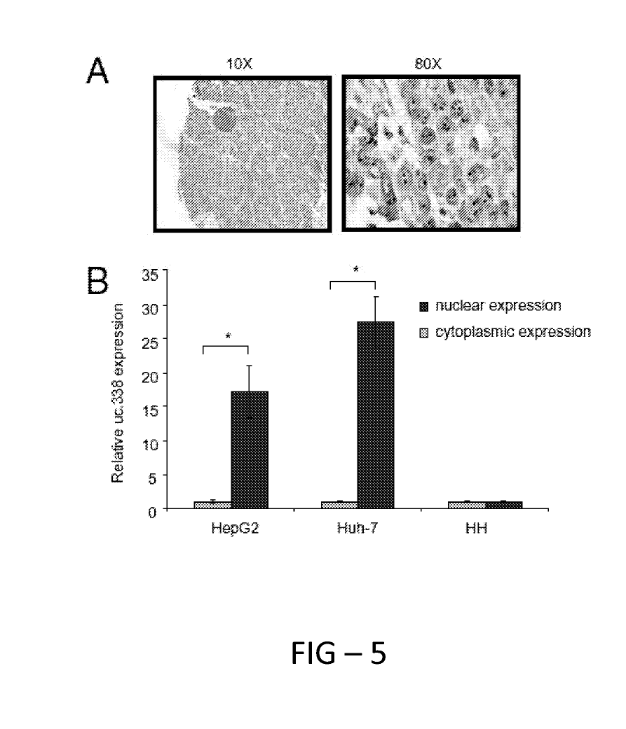

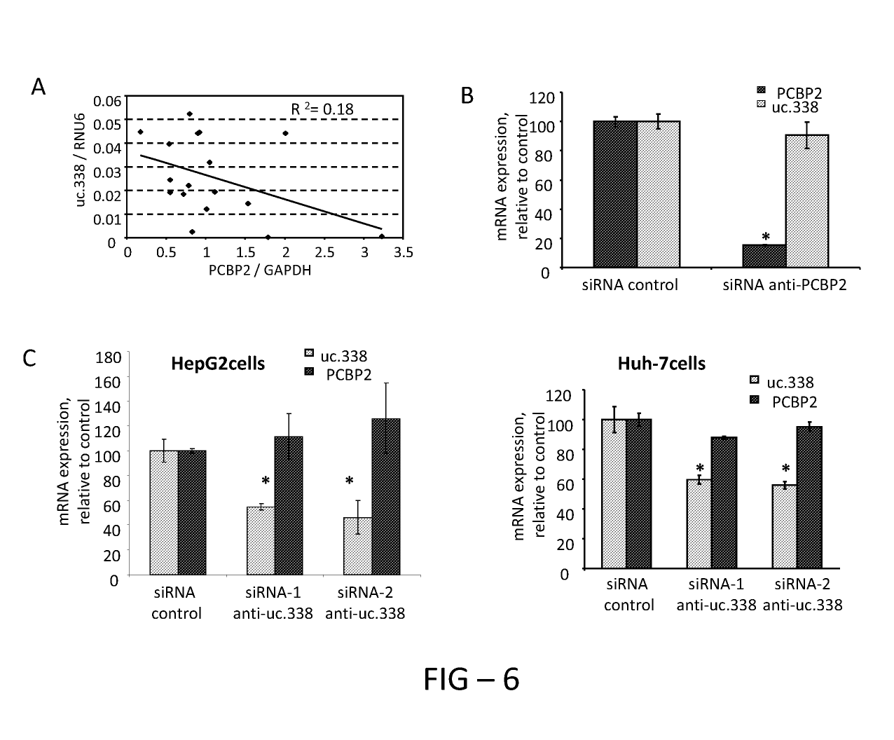

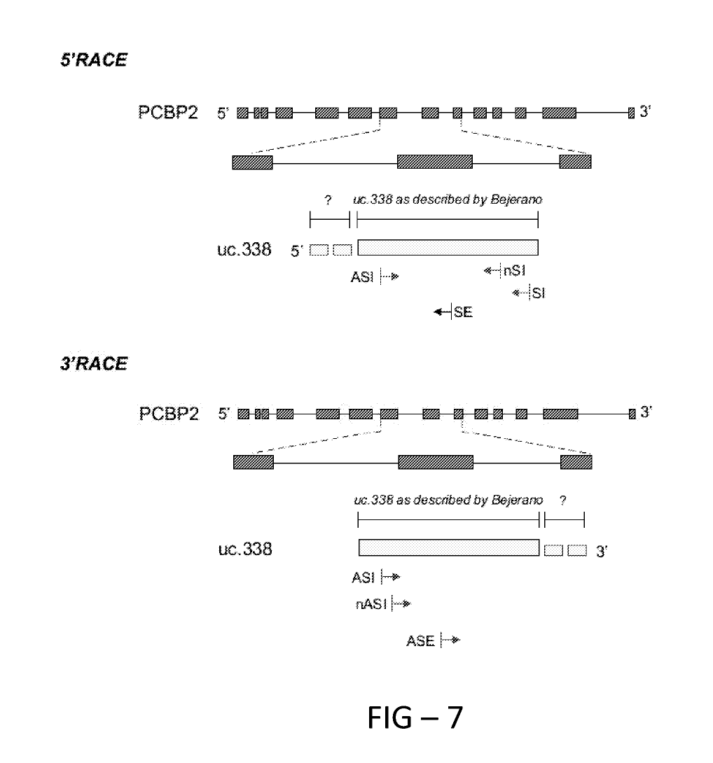

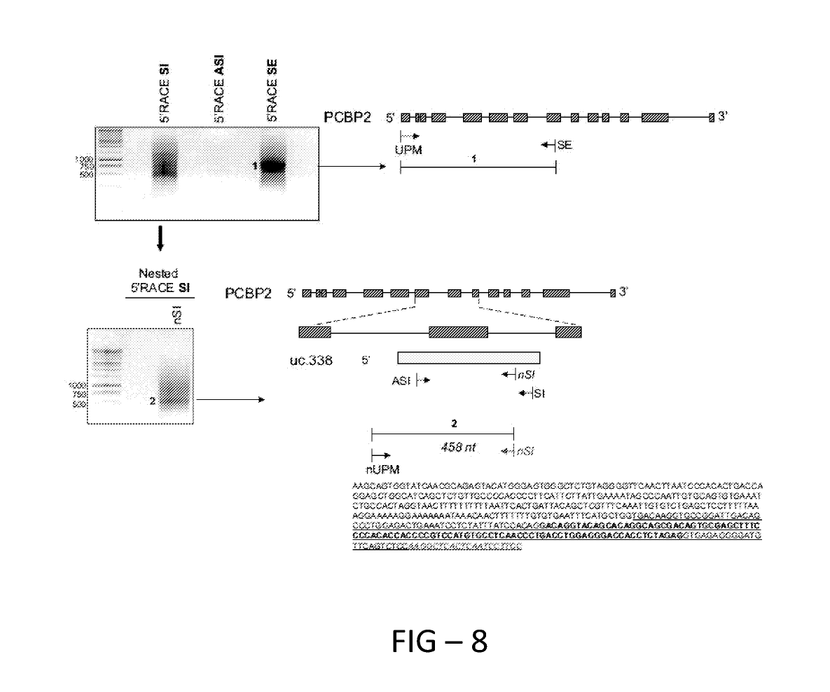

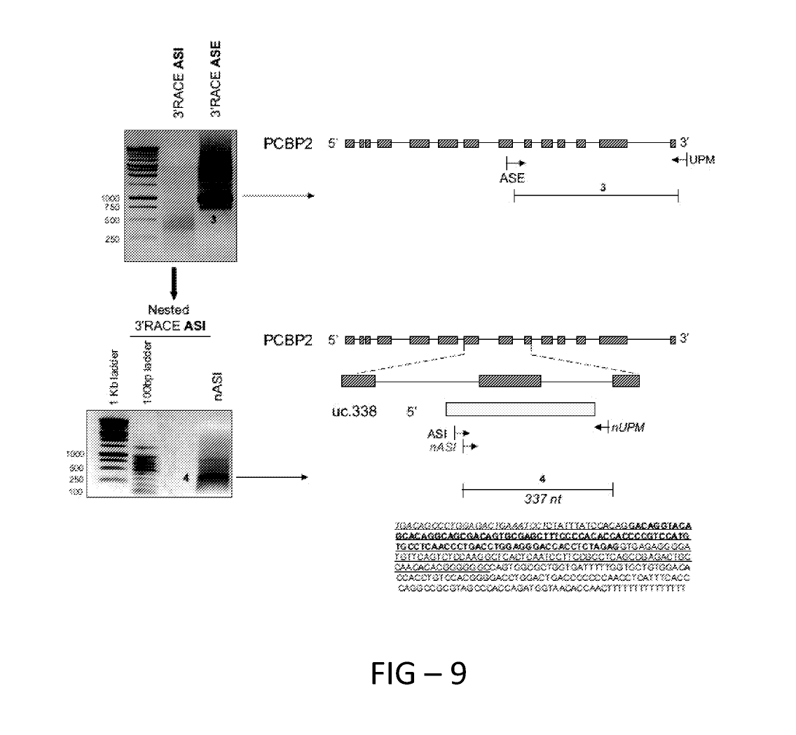

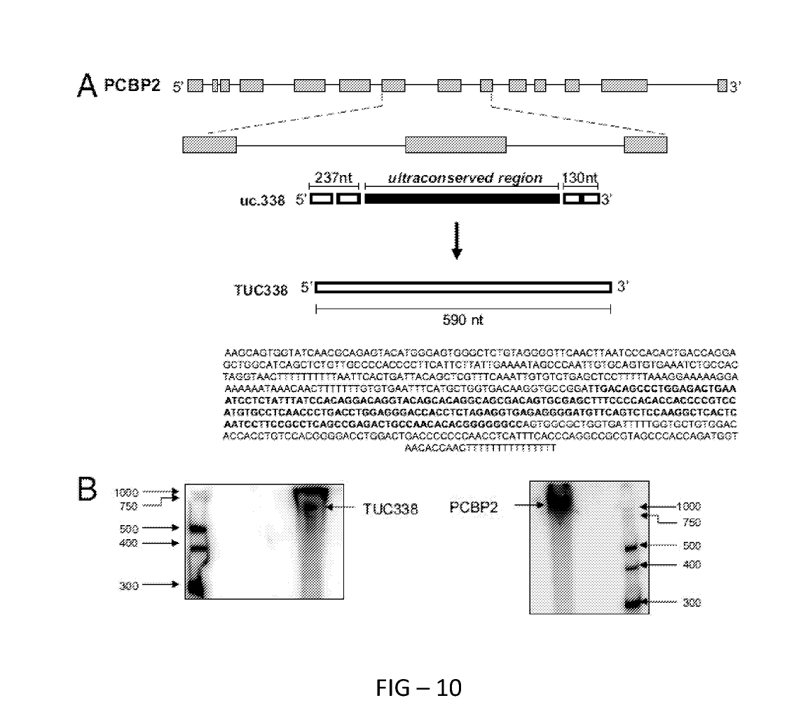

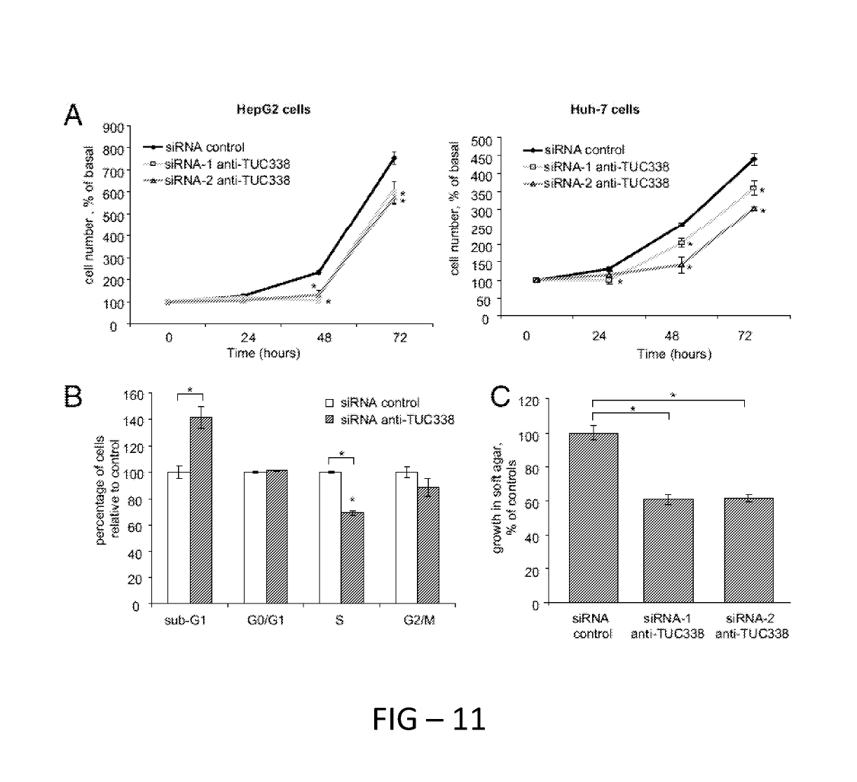

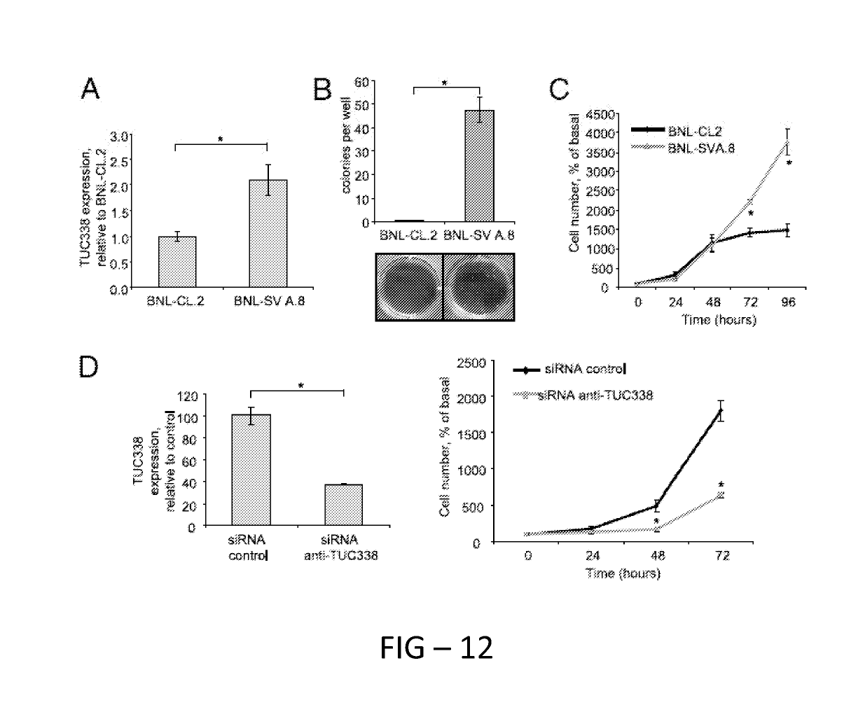

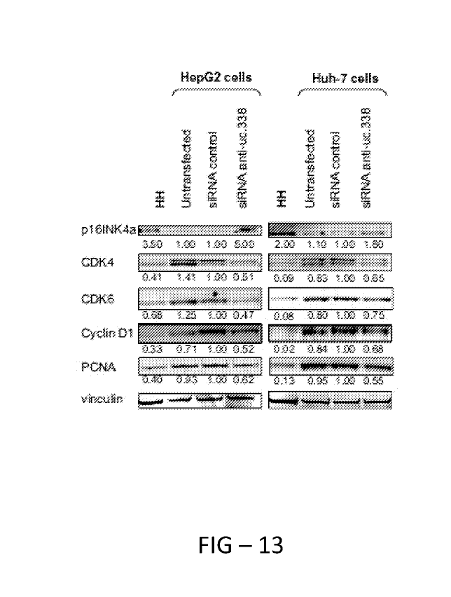

Aberrant expression of selected ucRNAs in malignant hepatocytes. Genome-wide expression profiling identified 56 ucRNAs, representing 11% of all ucRNAs analyzed, that were aberrantly and significantly (p<0.05) expressed in malignant HepG2 cells compared to non-malignant human hepatocytes (FIG. 1A). Of these, 33 were increased by 1.3 to 6.9-fold, whereas 23 were decreased by 0.8 to 0.3-fold in malignant hepatocytes (see Table 2). Exonic ucRNAs were not selectively enriched in malignant cells and the proportion of exonic regions in aberrantly expressed ucRNA (29% exonic, 42% non-exonic) was similar to those of all ultraconserved regions (FIG. 1B). The greatest change was noted for uc.338, which was increased in expression by 6.9±0.9 fold (p=0.001), and we thus focused our attention on this RNA gene.

uc.338 is increased in expression in HCC cell lines. In humans, uc.338 is an exonic ucRNA encoded within the gene PCBP2 in chromosome 12. uc.338 is comprised of 223 nucleotides, of which 93 nucleotides overlap with the coding sequence of PCBP2 (FIG. 2A). By real-time PCR, a striking increase in uc.338 expression by 2.2 to 5.1-fold was observed in several HCC cell lines compared to non-malignant human hepatocytes (FIG. 2B). We next determined uc.338 expression in a panel of human cancer cell lines, including biliary, pancreatic, colorectal, prostatic and breast cancers. In most cells, the expression of uc.338 was reduced or comparable to the expression in normal hepatocytes. Interestingly, all cholangiocarcinoma cells showed very low levels of uc.338 expression, suggesting that uc.338 may differentiate between primary liver cancers arising from different hepatic epithelia. Thus, uc.338 is increased in HCC cells and might be a promising marker for HCC.

uc.338 expression is increased in human HCC tissues. We next studied uc.338 expression in HCC tissues by in situ hybridization (ISH). 221 HCC samples in two tissue microarrays were analyzed. The arrays included 169 cases of adjacent non-cancerous liver tissue, with cirrhosis present in 97 cases. uc.338 expression was classified based on the percentage of cells with detectable expression as follows: negative (<5%), weak (5-19%), moderate (20-49%) or strong (≧50%). uc.338 expression was detected in 170 cases (77%), with a moderate to strong expression in 62% of these (FIG. 4A). The mean uc.338 expression was 4.0±1.5% in non-cirrhotic liver, 15.0±4.5% in cirrhotic liver and 24.0±5.7% in HCC tissues (FIG. 4B). Adequate paired tumoral and adjacent non-tumoral tissue for analysis was available from 156 cases. Of these, uc.338 expression was increased in 62%, was unchanged in 24% or was decreased in 14% of HCC compared to adjacent tissues (FIG. 4C). Consistent increases in uc.338 expression (score>2.0) were noted with HCC although a reduction in uc.338 expression (score<−2.0) occurred sporadically (FIG. 4D). Within tumor cells, uc.338 expression was predominantly nuclear (FIG. 5A). Similarly, the nuclear/cytoplasmic ratio of uc.338 expression in HepG2 and Huh-7 cells was 17 and 27 respectively (FIG. 5B).

uc.338 expression is regulated independently of PCBP2. uc.338 consists of 223 nt that are highly conserved throughout the species. In humans, the uc.388 ultraconserved region is located partly within the exon on the PCBP2 gene on chromosome 12. To evaluate the potential inter-relationships between PCBP2 and uc.338 transcription, we first examined the expression of PCBP2 by real time PCR in normal and HCC cell lines. The primers used spanned a genomic region in exons 10-13 of PCBP2 that was distant from that of uc.338 (FIG. 2A). PCBP2 expression was not increased in any of the HCC cell lines with the exception of Huh-7 cells, and PCBP2 expression did not correlate with that of uc.338 in all samples tested with a correlation coefficient of linear regression (R²) of 0.18 (FIG. 6A). Thus, uc.338 expression occurs independently of PCBP2 despite their overlapping genomic locations. uc.338 expression was unchanged in HepG2 cells transfected with siRNA against PCBP2 despite an 85% reduction in PCBP2 mRNA expression. (FIG. 6B). We next evaluated the effect of uc.338 on PCBP2 expression in HepG2 and Huh-7 cells but did not observe any effect of reduction in uc.338 expression on PCBP2 expression (FIG. 6C). Two siRNAs were tested. siRNA-1 reduced uc.338 expression by 46% in HepG2 and 40% in Huh-7 cells whereas siRNA-2 reduced uc.338 expression by 54% in HepG2 and 45% in Huh-7 cells compared to scrambled nucleotide control siRNA. Moreover, transfection of normal human hepatocytes with a vector expressing full-length uc.338 resulted in a 1.4-fold increase in PCBP2 expression despite a 32-fold increase in uc.338 expression. These observations confirm that PCBP2 expression is not functionally modulated by uc.338 expression.

Identification of Transcript Encoding uc.338. Having shown that uc.338 is transcribed independently of PCBP2, we proceeded to clone the transcript encoding this ultraconserved element. Rapid Amplification of cDNA Ends (“RACE”) was performed to characterize the 5′end and the 3′end of this transcript, which we termed “TUC338”. HepG2 RNA was retrotranscribed with the SMARTerScribeRT that exhibits terminal transferase activity upon reaching the end of an RNA template and adds residues to the first strand cDNA. The SMARTer oligo contains a terminal stretch of modified bases that anneal to the extended cDNA tail, allowing the oligo to serve as a template for the RT. Thus, a complete cDNA copy of the original RNA with an additional SMARTer sequence at the end is generated. To study TUC338 we used intronic primers that would not recognize PCBP2 coding sequence (FIG. 7). No products were produced with the antisense intronic (ASI) primer suggesting that TUC338 is not encoded in antisense. Conversely, a few bands were produced after amplification with sense intronic (SI) primer, with the larger bands being around 500 nt. A nested PCR with the nested sense intronic (nSI) primer produced a single defined band of around 500 nt that was further sequenced, leading to the characterization of the 5′end of TUC338. As control, we used the sense exonic (SE) primer that produced a >800-nt band by recognizing coding sequence PCBP2 (FIG. 8). These findings further showed that the TUC338 transcript is different from the PCBP2 transcript and characterized the 5′end of TUC338. The 3′ RACE studies identified 130 nucleotides at the 3′end downstream of the ultraconserved sequence (FIG. 9). In conclusion the uc.338 ultraconserved element is part of a 590 nt-long transcript, TUC388, that is transcribed independently on PCBP2 (FIG. 10).

Functional expression analysis of TUC338 regulated genes. To gain insight into the functional role of TUC338, we performed gene annotation enrichment analysis of genome-wide mRNAs that were changed in expression after TUC338 inhibition using siRNA. Functional annotation analysis identified the top four significantly over-represented cellular process gene classifications (and number of genes) as transcription (569), cell cycle (248), ubiquitin cycle (225) and cell division (115), whereas the top four over-represented molecular function classifications were ligase activity (159), protein binding (1,810), nucleotide binding (774), and ATP binding (638). The top four significantly over-represented GenMAPP pathway gene classifications were cell cycle-KEGG (56), mRNA processing reactome (63), RNA transcription reactome (28), and G1 to S cell-cycle reactome (41). These data suggested that TUC338 could modulate cellular processes involved in cell growth.

TUC338 modulates cell growth in human hepatocytes. We assessed anchorage-dependent cell growth after transfection with either siRNA to TUC338 or scrambled nucleotide control siRNA. Compared to control siRNA, siRNA-1 and siRNA-2 reduced cell proliferation by 21%, (p<0.001), or 24% (p=0.01) respectively, after 72 hours in HepG2 cells. Similar findings were observed in Huh-7 cells (FIG. 11A). Compared to controls there was a reduction of cells in S phase (p<0.0001) in cells transfected with siRNA to TUC338 (FIG. 11B). Cancer is characterized by the acquisition of cellular traits that enhance cell growth under adverse micro-environmental conditions. Therefore, we examined anchorage-independent growth by examining growth in soft agar assays. Compared to controls, siRNA to TUC338 reduced soft agar growth of HepG2 cells by 40.0±2.0% (FIG. 11C).

TUC338 modulates cell growth in mouse hepatocytes. The sequence conservation of ucRNAs across diverse species suggests that these genes may participate in essential roles that may be similar across species. To examine cross-species similarities in the effects of TUC338, we studied the effect of this gene in modulating transformed cell growth in murine cells. First, we examined the effect of cell transformation on TUC338 expression in BNL-CL.2 embryonic mouse hepatocytes. Compared to the parental BNL-CL.2 cells, the expression of TUC338 was increased by 2.1-fold in BNL-SVA.8 cells which are derived from BNL-CL.2 by SV40 transformation (FIG. 12A). BNL-SVA.8 cells have increased anchorage-dependent and anchorage-independent growth in soft agar compared to BNL-CL.2 cells (FIGS. 12, B & C). Knockdown of TUC338 in BNL-SVA.8 cells using siRNA caused a dramatic reduction in cell proliferation. At 72 hours, cell proliferation was reduced by 65.0±2.0% (p=0.0001) in BNL-SVA.8 cells transfected with siRNA to TUC338 compared to control siRNA (FIG. 12D). These data, indicating a relationship among TUC338 expression, cell transformation, and cell growth in mouse hepatocytes, are similar to those observed in humans.

TUC338 modulates progression through the cell cycle. The functional genomic expression analysis showed enrichment in genes involved in cell cycle progression from phase G1 to phase S in response to inhibition of TUC338. Moreover, inhibition of TUC338 in HCC reduced the number of cells in S phase (FIG. 11). Thus, we assessed the effect of TUC338 inhibition on expression of several proteins involved in the G1/S checkpoint in HepG2 and Huh7 cells. S phase progression can be modulated by CDK4/6-cyclin D1 mechanisms, and alterations in cyclin D are prominent in HCC. After inhibition of TUC338, we observed an increase in expression of the tumor suppressor p16INK4a and an associated reduction of CDK4, CDK6 and cyclin D1 (FIG. 13). Indeed, altered expression of these cell cycle regulatory proteins are associated with HCC growth: Although S-phase progression can also be modulated by the CDK2-cyclin E, effects on cell growth after inhibition of TUC338 were noted in Huh7 cells, which do not express p21 and cyclin E, making it unlikely that the effects of TUC338 on cell cycle progression involved these proteins.

Testing for uc.338 in human blood samples. It should be recognized that the above experiments can be done using blood samples. In such a circumstance, serum samples are obtained from individuals with hepatocellular cancer (HCC) or chronic liver disease without HCC. 50 fmol mmu-miR-295 mimics (Qiagen, Valencia, Calif.) are added into 100 μl serum and incubated for 5 minutes. RNA is then extracted using TRIZOL reagent (Invitrogen, Carlsbad, Calif.). Briefly, 1.0-mi TRIZOL reagent and 200-μl chloroform are added to the serum sample and the mixture is vortexed for 15 seconds and kept at 25° C. for 3 minutes. After centrifugation at 12,000 g for 15 minutes at 4° C., the supernatant is transferred to a fresh tube and 500-μl isopropanol is added. After incubation at −20° C. for 20 minutes, the mixture is centrifuged at 12,000 g for 10 minutes at 4° C. to remove the supernatant and the RNA pellet is washed with 75% ethanol. After removal of ethanol by centrifugation at 7500 g for 5 min at 4° C., RNA is air-dried for 5 minutes and then dissolved in 30-μl RNase-free water. Each sample of 11.5-μl RNA is polyadenylated and reversely transcribed to cDNA in a final volume of 30 μl using polyadenylation polymerase (New England Biolabs, Beverly, Mass.) and First-Strand cDNA Synthesis Kit with oligo-d(T) primer. The cDNA product is 1:5 diluted with water and stored at −80° C. for analysis. Real-time quantitative PCR (qPCR) quantification of TUC338 is performed using SYBR Green PCR Master Mixture. mmu-miR-295 is used as an internal normalization control. Melting curve analysis is performed at the end of PCR cycles in order to validate the specificity of the expected PCR product. All samples are run in duplicate, including blank controls without cDNA. The cycle threshold (Ct) is defined as the number of cycles required for the fluorescent signal to cross the threshold in qPCR. The formula 2^{ΔCt} is used to calculate the levels of TUC338 in serum, where ΔCt=mean (Ct of mmu-miR295)-Ct of TUC338.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of analyzing a biological specimen to detect cancer in a subject, comprising the steps of:
determining the expression level of an RNA sequence in the specimen;
comparing the determined expression level to a control, wherein a pre-identified difference between the determined expression level and the control is indicative of cancer in the subject.

2. The method of claim 1, wherein the RNA sequence is uc.338.

3. The method of claim 1, wherein the RNA sequence is selected from the group consisting of uc.338, uc.24, uc.189, uc.134, uc.378, uc.349, uc.78, uc.233, uc.262, uc.331, uc.136, and uc.246.

4. The method of claim 1, wherein the RNA sequence is selected from the group consisting of uc.110, uc.473, uc.275, uc.477, uc.269, uc.448, and uc.20.

5. The method of claim 2, wherein the subject is a mammal.

6. The method of claim 2, wherein the subject is a human.

7. The method of claim 6, wherein the cancer is a hepatocellular cancer.

8. The method of claim 7, wherein the control is a non-malignant hepatocyte.

9. The method of claim 2, wherein the step of determining the expression level includes the substep of determining the expression level by at least one of the following: an amplification assay and a hybridization assay.

10. The method of claim 1, wherein the RNA sequence is TUC338.

11. The method of claim 1, wherein the RNA sequence is for a transcript that is capable of encoding an ultraconserved RNA selected from the group consisting of uc.338, uc.24, uc.189, uc.134, uc.378, uc.349, uc.78, uc.233, uc.262, uc.331, uc.136, and uc.246.

12. The method of claim 1, wherein the RNA sequence is for a transcript that is capable of encodring an ultraconserved RNA selected from the group consisting of uc.110, uc.473, uc.275, uc.477, uc.269, uc.448, and uc.20.

13. The method of claim 11, wherein the subject is a mammal.

14. The method of claim 13, wherein the the subject is a human.

15. The method of claim 14, wherein the cancer is a hepatocellular cancer.

16. A tumor-suppressing agent comprising, as an active ingredient, at least one of:
a double-stranded RNA complementary to a transcript of an ultraconserved RNA gene; a DNA encoding a double-stranded RNA complementary to a transcript of an ultraconserved RNA gene; and a venctor carrying, as an insert, a DNA encoding a double-stranded RNA complementary to a transcript of an ultraconserved RNA gene.

17. The tumor-suppressing agent of claim 16, wherein the ultraconserved RNA gene is selected from the group consisting of uc.338, uc.24, uc.189, uc.134, uc.378, uc.349, uc.78, uc.233, uc.262, uc.331, uc.136, and uc.246.

18. The tumor-suppressing agent of claim 16, wherein the ultraconserved RNA gene is selected from the group consisting of uc.110, uc.473, uc.275, uc.477, uc.269, uc.448, and uc.20.

19. The tumor-suppressing agent of claim 16, wherein the transcript is TUC338.

20. An anti-cancer composition comprising:
an active ingredient that binds to uc.338, the active ingredient selected from one of the following: an antisense oligonucleotide, ribozyme, siRNA, or any combination thereof.
